(19) United States
(12) Patent Application Publication
(10) Pub. No.: US 2013/0039864 A1
(43) Pub. Date: Feb. 14, 2013

(54) Non-Intravenous Dosage Form Comprising Solid Formulation of Liquid Biologically Active Agent and Uses Thereof

(71) Applicants: Francois Ravenelle, Montreal (CA); Dorothee Le Garrec, Montreal (CA); David Lessard, Montreal (CA); Sandra Gori, Montreal (CA); Damon Smith, Saint-Laurent (CA); Miloud Rahmouni, Pierrefonds (CA); Vinayak Sant, Pittsburgh, PA (US)

(72) Inventors: Francois Ravenelle, Montreal (CA); Dorothee Le Garrec, Montreal (CA); David Lessard, Montreal (CA); Sandra Gori, Montreal (CA); Damon Smith, Saint-Laurent (CA); Miloud Rahmouni, Pierrefonds (CA); Vinayak Sant, Pittsburgh, PA (US)

(21) Appl. No.: 13/643,038

(22) PCT Filed: Apr. 21, 2011
(86) PCT No.: PCT/CA11/00447

§ 371 Date: Oct. 23, 2012

Related U.S. Application Data
(60) Provisional application No. 61/327,348, filed on Apr. 23, 2010.

(51) Int. Cl. A61K 47/34; A61K 9/68; A61P 25/00; A61P 1/08; A61P 23/00; A61P 25/20; A61P 25/06; A61P 25/02; A61K 31/05; A61P 25/04; B82Y 40/00; B82Y 5/00

(52) U.S. Cl. 424/48; 514/731; 514/772.1; 977/788; 977/906; 977/840

(57) ABSTRACT

The disclosure relates to a non-intravenous dosage for administration of a liquid biologically active agent. The dosage form contains a solid formulation of the liquid biologically active agent, e.g. propofol, in intimate association with at least one stabilizing agent, e.g. an amphiphilic polymer or surfactant. A liquid biologically active agent is converted to a solid product, e.g. a powder, that can be easily incorporated into a number of different non-intravenous dosage forms. Upon hydration, a nanodispersion or micelle loaded with the active agent is formed. The dosage form can provide a non-intravenous route of administration for active agents that are typically only administered intravenously. Methods, uses, kits and commercial packages related to the non-intravenous dosage form are also disclosed.

### CROSS REFERENCE TO RELATED APPLICATIONS

This application claims the benefit of priority of U.S. Provisional Patent Application No. 61/327,348 filed Apr. 23, 2010, which is incorporated herein by reference in its entirety.

### TECHNICAL FIELD

The present disclosure relates to a non-intravenous dosage form comprising a solid formulation of a liquid biologically active agent, wherein the solid formulation comprises the liquid biologically active agent in intimate association with at least one stabilizing agent. The disclosure further relates to formulations, methods, uses, kits and commercial packages pertaining to the non-intravenous dosage form.

### BACKGROUND

A variety of methods and procedures have been described in the prior art for preparing stable formulations for the effective delivery of hydrophobic and amphiphilic biologically active agents to a desired location in the body. A number of these methods are based on the use of auxiliary solvents; surfactants; soluble forms of the drug, e.g., salts and solvates; chemically modified forms of the drug, e.g., prodrugs; soluble polymer-drug complexes; special drug carriers such as liposomes and micelles; and others. These methods and procedures generally result in formulations intended for intravenous administration. Furthermore, many of the above methods and procedures have drawbacks related to such factors as toxicity, poor entrapment, relatively large particle sizes, or the time and cost associated with the materials or method of preparation.

There have also been attempts investigating the use of water-soluble prodrugs, for example, to provide options for oral dosage forms. However, often prodrugs require much higher doses for the same response and usually demonstrate a slower onset of action and slower clearance, which can be a disadvantage where rapid drug action is required. Prodrugs are often unstable resulting in short shelf lives or low storage temperatures to maintain their stability.

Polymeric and surfactant-based micelles and nanodispersions are being heavily investigated as carriers of poorly water-soluble molecules. Micelles demonstrate a core-shell structure that allows the active agent to be protected during transportation to the target site. The hydrophobic inner core generally serves as a microenvironment for the solubilization of the active agent, whereas the hydrophilic outer shell is responsible for micelle stability and aqueous stability.

Polymeric micelles are discussed in, for example, Jones and Leroux, Eur. J. Pharm. Biopharm. (1999) 48, 101 111; Kwon and Okano, Adv. Drug Deliv. Rev. (1996) 21, 107-116 and Allen et al. Colloids Surf. B: Biointerf. (1999) 16, 3-27. Pharmaceutical research on polymeric micelles has been mainly focused on copolymers having an AB diblock structure with A representing the hydrophilic shell moieties and B representing the hydrophobic core polymers, respectively. Multiblock copolymers such as poly(ethylene oxide)-poly(propylene oxide)-poly(ethylene oxide) (PEO-PPO-PEO) (A-B-A) can also self-organize into micelles, and have been described as potential drug carriers, e.g. Kabanov et al., FEBS Lett. (1989) 258, 343-345.

The hydrophobic core which generally consists of a biodegradable polymer such as a poly(β-benzyl-aspartate) (PBLA), poly(D,L-lactic acid) or poly(ε-caprolactone), serves as a reservoir for a poorly water-soluble drug, protecting it from contact with the aqueous environment. The core may also consist of a water-soluble polymer, such as poly(aspartic acid) (P(Asp)), which is rendered hydrophobic by the chemical conjugation of a hydrophobic drug, or is formed through the association of two oppositely charged polyions (PICM). Several studies also describe the use of poorly- or non-biodegradable polymers, such as polystyrene (PSI) or poly(methyl methacrylate)(PMMA), as constituents of the inner core. See, e.g., Zhao et al., Langmuir (1990) 6, 514-516; Zhang et al., Science (1995) 268, 1728-1731; Inoue et al., J. Controlled Release (1998) 51, 221-229 and Kataoka J. Macromol. Sci, Pure Appl. Chem. (1994)A31, 1759-1769. The hydrophobic inner core can also consist of a highly hydrophobic small chain such as an alkyl chain or a diacyllipid (e.g. distearoyl phosphatidyl ethanolamine). The hydrophobic chain can be either attached to one end of a polymer, or randomly distributed within the polymeric structure. The shell usually consists of chains of hydrophilic, non-biodegradable, biocompatible polymers such as poly(ethylene oxide) (PEO) (see Allen et al. Colloids Surf. B: Biointerf. (1999) 16, 3-27 and Kataoka et al. J. Controlled Release (2000) 64, 143-153), poly(N-vinyl-2-pyrrolidone) (PVP) (see Benahmed A et al. Pharm Res (2001) 18, 323-328) or poly(2-ethyl-2 15 oxazoline) (see Lee et al. Macromolecules (1999) 32, 1847-1852).

In general, polymeric micelles have been investigated for intravenous delivery of biologically active agents and are not generally contemplated for non-intravenous routes of administration. Furthermore, polymeric micelles are generally used in the delivery of biologically active agents that are solids. However, a number of important biologically active agents are liquid, for example, propofol.

Propofol (2,6-bis-(1-methylethyl)phenol, or 2,6-diisopropylphenol) is one of the most popular anesthetics in the world. It is most commonly used for the induction and maintenance of anaesthesia or sedation upon intravenous (i.v.) administration to humans or animals.

Propofol is an oil that is immiscible with water (aqueous solubility of approximately, 0.154 mg/mL); its is commonly supplied in the form of an emulsion, at concentrations of 1% or 2% (w/w), with 2% being used for longer sedation. Propofol oil-in-water emulsions currently on the market include DIPRIVAN® (manufactured by AstraZeneca Pharmaceuticals, Inc.), BAXTER® IPP (manufactured by Gensia Sicor, Inc.), and a propofol injectable emulsion manufactured by Bedford Laboratories. These are all formulated for intravenous administration.

WO 06/056064 (Ravenelle et al.) describes a solid formulation of propofol that is reconstituted, prior to intravenous administration, to form a clear, stabilized, nanodispersion or loaded micelles comprising a polymer as a stabilizing agent. However, there is no mention of non-intravenous administration.

When orally administered as a homogeneous liquid suspension, propofol is reported to exhibit an oral bioavailability of about 5% that of an equivalent intravenous dose of propofol. It is because of its poor oral bioavailability and extensive first-pass metabolism, that propofol is currently administered by injection for intravenous infusion only. Oral administration of propofol has not been considered therapeutically effective and has not been possible with the formulations currently available. This has prevented investigations into the efficacy of propofol for treating diseases or conditions for which intravenous infusion is not appropriate, such as diseases and conditions benefiting from outpatient treatment or where intravenous infusion is not possible or suitable. Thus, despite the widespread use of propofol, it currently has little value in these settings.

While the main clinical use of propofol is anaesthesia, there is emerging evidence that propofol is useful in the treatment and prevention of headache (e.g. migraine or cluster headache), nausea and emesis. There are a number of patients who suffer from intractable migraine headache, nausea and emesis who are not served by current medications. However, since propofol is only available as an i.v. injection for anaesthesia, it is not suitable for these conditions.

Examples of treatments for migraine using propofol are disclosed in the following references: Propofol: A New Treatment Strategy for Refractory Migraine Headache, Jacqueline Drummond-Lewis and Corey Scher, Pain Medicine, Volume 3, Number 4, 2002, 366-369; Intravenous Propofol: Unique Effectiveness in Treating Intractable Migraine, John Claude Krusc et al., Headache, 2000; 40: 224-230; Intravenous Propofol in the Treatment of Refractory Headache, Headache, 2002; 41-638-641. Krusz J. C. et al (Headache 2000; 40: 224-230) describe the efficacy of intravenous propofol in treating intractable migraine. It will be noted that all these treatments are all intravenous.

Recent studies have demonstrated the efficacy of propofol in treating emesis and intractable migraines when administered intravenously at sub-sedative, sub-hypnotic doses. In the treatment of emesis, propofol has been used mostly with cancer patients who receive chemotherapy, and the normal treatment is usually by the intravenous route (A. Borgeat, O. H. G. Wilder-Smith and M. Formi, Canadian journal of anaesthesia, 40(69), 1993). This is normally achieved through propofol premedication prior to chemotherapy treatments to prevent symptoms. For example, propofol has been used at subhypnotic doses (0.5-1 mg/kg/h) for the prevention and treatment of chemotherapy induced emesis (Borgeat et al. Oncology 1993; 50: 456-459; Scher C S et al. Canad. J. Anaesth. 1992; 39: 170-2) and of postoperative emesis (Borgeat A. et al. Anaesthesia and Analgesia 1992; 74: 539-41, and Schulman S R et al. Anaesthesia and Analgesia 1995; 80: 636-37).

Propofol has been used to control cancer pain in patients (Hooke et al., J Ped Oncology Nursing 2007, 24(1), 29-34), and in pre-clinical studies, locally injected propofol produces an antinociceptive effect in an animal models of inflammatory pain (Guindon et al., Anesth Analg 2007, 104, 1563-1569). Propofol has also been shown to be effective in the treatment of central pain such as trigeminal neuralgia (Kubota et al., Exp Brain Res. 2007, 179(2), 181-190; and Mizuno et al., Neurol Med Chir (Tokyo) 2000, 40(7), 347-50), spinal cord injury (SCI) pain (Canavero and Bonicalzi, Neurol Sci 2001, 22, 271-273; and Canavero and Bonicalzi, Clin Neuropharmacol 2004, 27(4), 182-186), and central post-stroke pain (CPSP) (Canavero et al., J Neurol 1995, 242(9), 561-567; and Canavero and Bonicalzi, Pain 1998, 74(2-3), 109-114).

There is a need for alternative formulations of hydrophobic or amphiphilic liquid biologically active agents capable of achieving levels of bioavailability sufficient for efficacy. In particular, there is an unmet need for non-intravenous dosage forms, such as oral, sublingual, intranasal, intrapulmonary, rectal, urethral, vaginal, ocular, otic, or topical dosage forms, suitable for use in a hospital or outpatient setting. One example of an important hydrophobic liquid biologically active agent is propofol. While there is evidence that propofol is effective in treating intractable migraine, headache, nausea, vomiting, and pain, the current dosage forms are for i.v. administration only and therefore are not suitable for outpatient use. Thus, there remains an unmet need for new propofol dosage forms that can be administered conveniently to patients in a non-intravenous manner.

### SUMMARY

It is an object of the present disclosure to obviate or mitigate at least one disadvantage of previous formulations comprising liquid biologically active agents.

In a first aspect, there is provided a dosage form for non-intravenous administration of a liquid biologically active agent. The dosage form comprising a solid formulation comprising the liquid biologically active agent in intimate association with at least one stabilizing agent. The dosage form may further comprise one or more additives.

The dosage form, upon hydration, is capable of forming a nanodispersion or micelle loaded with the liquid biologically active agent.

The stabilizing agent may comprising at least one amphiphilic copolymer or at least one surfactant. The amphiphilic copolymer may comprise a linear, branched or star-shaped block polymer.

In some embodiments, the amphiphilic polymer includes a hydrophilic segment is selected from poly(ethylene oxide), poly(N-vinylpyrrolidone), poly(N-2-hydroxypropylmethacrylamide), poly(2-ethyl-2-oxazoline), poly(glycidol), poly(2-hydroxyethylmethacrylate), poly(vinylalcohol), polymethacrylic acid derivatives, poly(vinylpyridinium), poly((ammoniumalkyl)methacrylate), poly((aminoalkyl)methacrylate) and combinations and derivatives thereof; and a hydrophobic segment selected from the group comprising a poly(ester), poly(ortho ester), poly(amide), poly(esteramide) poly(anhydride), poly(propylene oxide), poly(tetrahydrofuran), polystyrene, polymethacrylate, polyacrylate, polymethacrylic acid, polyacrylic acid and combinations and derivatives thereof.

In some embodiments, the hydrophobic segment comprises a poly(ester) selected from the group consisting of poly(ε-caprolactone), poly(lactide), poly(glycolide), poly(lactide-co-glycolide), poly(hydroxyl-alkanoates), poly(β-malic acid), and combinations and derivatives thereof.

In some embodiments, the amphiphilic copolymer is a PVP-PDLLA or PEG-PMA copolymer. The amphiphilic copolymer may, for example, be a diblock or triblock PEG-PMA copolymer. In some embodiments, the PEG-PMA copolymer is an EG-MAA-BMA copolymer having the composition: EG₍₂₀₋₅₀₀₎-MAA₍₅₋₅₀₀₎-BMA₍₅₋₅₀₀₎, which may include polymers having the following compositions: EG₍₄₅₎-MAA₍₆₃₎-BMA₍₂₈₎; EG₍₄₅₎-MAA₍₆₄₎-BMA₍₃₄₎; or EG₍₄₅₎-MAA₍₅₄₎-BMA₍₂₆₎.

In some embodiments, the amphiphilic copolymer is a PVP-PDLLA copolymer.

In some embodiments, the stabilizing agent comprises a surfactant, such as, lauryl sulphate, hexadecyl pyridinium chloride, polysorbates, sorbitans, poly(oxyethylene) alkyl ethers, poly(oxyethylene) alkyl esters and combinations thereof.

In some embodiments, the dosage form is prepared from a solid formulation comprising the liquid biologically active agent in intimate association with at least one stabilizing agent, and one or more additives. In some embodiments, the solid formulation is obtained by drying a mixture of the stabilizing agent, the liquid biologically active agent, and at least one solvent therefore, in such a manner as to form the intimate mixture of the liquid biologically active agent and the stabilizing agent. The drying may be lyophilization or freeze-drying. In some embodiments, the drying results in a powder, which may the involve spray-drying or fluid bed-drying.

wherein the liquid biologically active agent is present in the solid formulation in a therapeutically effective amount.

In some embodiments, the liquid biologically active agent is present in the solid formulation in an amount between about 1 wt % and about 80 wt %, between about 1 wt % and about 60 wt %, between about 5 wt % and about 40 wt %, between about 5 wt % and about 30 wt %, between about 10 wt % and about 30 wt %, between about 10 wt % and about 20 wt %, between about 0.1 wt % and 5 wt %, between about 1 wt % and about 5 wt %.

In some embodiments, the solid formulation is present in the dosage form in an amount from about 1 wt % to about 99 wt %, from about 5 wt % to about 85 wt %, from about 5 wt % to about 60 wt %, 5 wt % to about 40 wt %, between about 5 wt % to about 30 wt %, between about 10 wt % to about 30 wt %, between about 10 wt % to about 20 wt %, between about 0.1% to 5%, between about 1 wt % to about 5 wt %, between about 20 wt % to about 60 wt %.

In some embodiments, the biologically active agent is present in the dosage form in an amount from about 0.01 wt % to about 80 wt %, 0.01 wt % to about 50 wt %, from about 1 wt % to about 20%, from about 1 wt % to about 15 wt %, from between about 2 wt % to about 10 wt %, between about 1 wt % to about 5 wt %, between about 5 wt % to about 10 wt %, or between about 10 wt % to about 20 wt %.

In some embodiments, the dosage form provides a bioavailability sufficient for achieving therapeutic efficacy. In some embodiments, the bioavailability of the active agent is at least about 2%, 5%, 10%, 15%, 20%, 25%, 30%, 35%, 40%, 45%, 50%, 55%, 60%, 65%, 70%, 75%, 80%, 85%, 90%, 95%, 100%, or higher. In some embodiments, the dosage form exhibits an increase in bioavailability of at least 10% compared to same-route administration of the biologically active agent in the absence of the stabilizing agent. In some embodiments, the dosage form exhibits a relative bioavailability of at least 100%, 110%, 120%, 150%, 200%, 500%, 700%, or 1000%. In some embodiments, the dosage form exhibits a absolute bioavailability of at least 10%. In some embodiments, the bioavailability of the active agent is increased by at least about 1.5-fold, 2-fold, 3-fold, 5-fold, 10-fold, 15-fold, 20-fold, 30-fold, 40-fold, 50-fold, 75-fold, 100-fold, or higher, in the presence of the stabilizing agent. In some embodiments, the bioavailability of the active agent is increased by at least about 1.5-fold to about 40-fold, from about 2-fold to about 35-fold, from about 5-fold to about 30-fold, in the presence of the stabilizing agent.

In some embodiments, the solid formulation has a drug loading level (DLL) of up to about 5%, 10%, 15%, 20%, 25%, 50%, 60%, 70%, 80%, or higher. In some embodiments, the solid formulation has a drug loading level (DLL) from about 1% to about 80%, from about 10% to about 80%, or from about 20% to about 60%.

In some embodiments, the solid formulation forms micelles having a diameter less than about 500 nm, such as, between about between about 5 nm to 500 nm, 10 nm to 500 nm, 10 nm to 400 nm, 20 nm to 300 nm, or 20 nm to 200 nm.

In some embodiments, the stabilizing agent has a CAC below about 100 mg/L, below about 50 mg/L, below about 25 mg/L, below about 10 mg/L, or below about 5 mg/L. In some embodiments, the stabilizing agent has a CAC in the range of about 0.1 mg/L to about 1000 mg/L, about 0.1 mg/L to about 100 mg/L, about 0.1 mg/L to about 50 mg/L, about 0.1 to about 25 mg/L, about 0.1 to about 10 mg/L, or about 0.1 to about 5 mg/L.

In some embodiments, the liquid biologically active agent is hydrophobic or amphiphilic. In some embodiments, the liquid biologically active agent is selected from the group consisting of propofol, quinaldine, methoxyflurane, nicotine, phytonadione, methoxyflurane, dinoprost tromethamine, and mesoprostol, or a prodrug or derivative thereof.

In some embodiments, the dosage form is suitable for oral, sublingual, intranasal, intrapulmonary, rectal, urethral, vaginal, ocular, otic or topical administration.

In some embodiments, it suitable for oral administration and may exhibit an absolute bioavailability of at least 10%. In some embodiments, it is suitable for sublingual administration.

In some embodiments, the dosage form is in the form of a tablet, caplet, capsule, sachet, solution, suspension, emulsion, cream, gel, film, lozenge, chewing gum, paste, ointment, drop, spray, aerosol inhaler, dry powder inhaler, suppository, pessary, or enema.

In some embodiments, the additive is one or more of a carrier, a bulk forming agent, a cryoprotectant, a lyoprotectant, a binder, a flavoring agent, a taste masking agent, a coloring agent, an odorant, a buffer, a preservative, a diluent, a dispersant, a surfactant, a disintegrant, or an additional stabilizer.

In some embodiments, tablet is a rapid disintegrating tablet (RDT). In some embodiments, the RDT comprises a disintegrant or disintegrating matrix to facilitate rapid release of the solid formulation from the dosage form. In some embodiments, the disintegrating matrix is a starch or a hydrogel. In some embodiments, the starch is a cross-linked high amylose starch, such as, Contramid. In some embodiments, the RDT additionally comprises a sugar, such as, mannitol, trehalose, maltodextran.

The dosage form may be an instant release dosage form, an immediate release dosage form, or a controlled release dosage form. In some embodiments, the dosage form is a controlled release dosage form and the controlled release is sustained release, and wherein the dosage form releases the liquid biologically active agent over a period of about 45 minutes to about 24 hours.

In some embodiments, the dosage form releases the liquid biologically active agent over a period of at least about 4 hours, at least about 8 hours, at least about 12 hours, at least about 16 hours, at least about 20 hours, or at least about 24 hours.

In some embodiments, the liquid biologically active agent is propofol or a derivative or prodrug thereof. In some embodiments, the liquid biologically active agent is propofol. In some embodiments, the solid formulation comprises between about 10 wt % and about 30 wt % propofol. In some embodiments, upon oral administration, the absolute bioavailability of propofol is at least about 10%, between about 15% and about 165%, between about 15% and about 100%, between about 15% and about 80%, or between about 20% and about 80%.

In some embodiments, dosage form is for use in the treatment or prevention of a disease or condition of the central nervous system. In some embodiments, condition of the central nervous system is headache, emesis, nausea, or pain

In some embodiments, dosage form is for inducing anaesthesia or sedation in a subject in need thereof. In some embodiments, the dosage form is for use in the manufacture of a medicament.

In another aspect, there is provided a use of a dosage form as described herein in the manufacture of a medicament for the treatment or prevention of a disease or condition of the central nervous system.

In another aspect, there is provided a use of a dosage form as described herein in the treatment or prevention of a disease or condition of the central nervous system.

In another aspect, there is provided a use of a dosage form as described herein in the manufacture of a non-intravenous dosage form for the treatment or prevention of a disease or condition of the central nervous system.

In another aspect, there is provided a use of a solid formulation comprising an intimate mixture of propofol and at least one amphiphilic copolymer in the manufacture of a non-intravenous dosage form for the treatment or prevention of a disease or condition of the central nervous system.

In another aspect, there is provided a solid formulation comprising an intimate mixture of propofol and at least one stabilizing agent, for use in the manufacture of a non-intravenous dosage form for the treatment or prevention of headache, nausea, emesis, or pain.

In another aspect, there is provided a method or treating a disease or condition, comprising administering to a subject in need thereof a therapeutically effective amount of a non-intravenous dosage form as described herein. In some embodiments, the route of administration is oral, sublingual, intranasal, intrapulmonary, rectal, urethral, vaginal, ocular or topical administration In some embodiments, the route of administration is oral administration. In some embodiments, the route of administration is sublingual administration.

In some embodiments, the disease or condition to be treated is a disease or condition of the central nervous system. In some embodiments, the disease or condition of the central nervous system is headache, nausea, emesis or pain. In some embodiments, the headache is intractable migraine headache. In some embodiments, the pain is neuropathic pain. In some embodiments, the neuropathic pain is post-herpetic neuralgia, peripheral neuropathy, trigeminal neuralgia, lower back pain, painful diabetic neuropathy, HIV-related neuropathic pain, cancer-related pain, or fibromyalgia.

In another aspect, there is provided a method of treating or preventing headache, nausea, emesis or pain, comprising administering to a subject in need thereof a therapeutically effective amount of a non-intravenous dosage form comprising a solid formulation, and, optionally, one or more additives, the solid formulation comprising an intimate mixture of propofol and at least one amphiphilic copolymer, wherein, upon hydration, micelles loaded with the propofol are formed.

In another aspect, there is provided a commercial package or kit comprising a non-intravenous dosage form as described herein, together with one or more instructions for use in the treatment or prevention of a disease or condition.

In another aspect, there is provided a commercial package or kit comprising a non-intravenous dosage form as described herein comprising propofol, together with one or more instructions for use in the treatment or prevention of headache, nausea, emesis, or pain.

In another aspect, there is provided a method for the preparation of a dosage form for non-intravenous administration of a liquid biologically active agent which comprises: providing a first mixture of at least one stabilizing agent in at least one solvent, under conditions to achieve micelle or nanodispersion formation, providing a second mixture by mixing said first mixture and at least one liquid biologically active agent to load said micelle or nanodispersion with said liquid biologically active agent, removing the solvent from said second mixture to form a solid formulation; and optionally, adding one or more additives suitable to prepare the non-intravenous dosage form.

In some embodiments, the solvent is removed by drying. In some embodiments, the drying involves spray drying or drying in a fluid bed. In some embodiments, the drying freeze drying.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a FIG. 1 illustrates the in vitro translocation of propofol from micellar formulations across Caco-2 monolayers.

FIG. 2 illustrates the pharmacokinetic profiles of Diprivan® IV (3.5 mg/kg), PM1a, PM1b (7 mg/kg, PO), and PM3 FD (35 mg/kg, PO) after administration to female Sprague-Dawley rats.

FIG. 3 illustrates the total exposure (AUC) after administration of Diprivan® IV (3.5 mg/kg), PM1a, PM1b (7 mg/kg, PO), and PM3 FD (35 mg/kg, PO) to female Sprague-Dawley rats.

FIG. 4 illustrates the pharmacokinetic profiles of Diprivan® IV (7 mg/kg), PM1c (7 mgkg), and PM1c (3.5, 7 and 14 mg/kg, PO) after administration to female Sprague-Dawley rats.

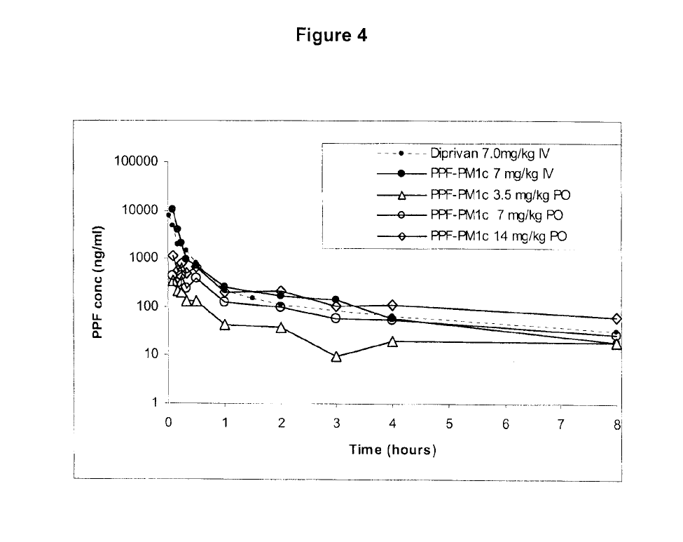

FIG. 5 illustrates the total exposure (AUC) after administration of PM1c (3.5, 7 and 14 mg/kg, PO) to female Sprague-Dawley rats.

FIG. 6 illustrates the pharmacokinetic profiles of Diprivan® IV (7 mg/kg) and PM2 (3.5, 7 and 14 mg/kg, PO) after administration to female Sprague-Dawley rats.

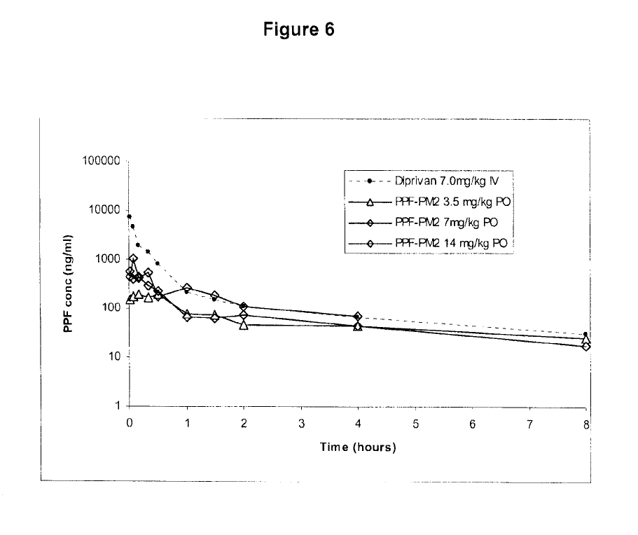

FIG. 7 illustrates the total exposure (AUC) after administration of PM2 (3.5, 7 and 14 mg/kg, PO) to female Sprague-Dawley rats.

FIG. 8 illustrates the pharmacokinetic profiles of Rapinovet® IV (mg/kg), PM3 FD (5 mg/kg, PO), and PM5 SD (3, 5 and 15 mg/kg, PO) after administration to male Göttingen Minipigs.

### DETAILED DESCRIPTION

Generally, the present disclosure provides a solid formulation of a liquid biologically active agent suitable for non-intravenous administration to a subject. The solid formulation comprises an intimate mixture of a liquid biologically active agent and at least one stabilizing agent, for example, an amphiphilic copolymer or a surfactant. The formulation may be used to improve the bioavailability of the liquid biologically active agent. Conveniently, the solid formulation permits the incorporation of the liquid biologically active agent into several different dosage forms suitable for non-intravenous administration to a human or animal. Various dosage forms, methods, uses, kits and commercial packages comprising the solid formulation are described herein, following the below definitions of abbreviations and terms used throughout the specification.

### Abbreviations

As used herein, the abbreviation n-BMA refers to n-butyl methacrylate.

As used herein, the abbreviation t-BMA refers to t-butyl methacrylate.

As used herein, the abbreviation PEGME refers to poly(ethyleneglycol) methyl ether.

As used herein, the abbreviation THF refers to tetrahydrofuran.

As used herein, the abbreviation PPF refers to propofol.

As used herein, the abbreviation PVP-PDLLA refers to polyvinyl pyrrolidone-polylactide block copolymers.

As used herein, the abbreviation PEG-PMA refers to poly(ethyleneglycol)-poly(methacrylate-co-methacrylic acid) block copolymers

As used herein, the abbreviation DDL refers to drug loading level

As used herein, the abbreviation SLS refers to static light scattering.

As used herein, the abbreviation NMR refers to nuclear magnetic resonance.

As used herein, the abbreviation Mn refers to number average molecular weight.

As used herein, the abbreviation Mw refers to weight average molecular weight.

As used herein, the abbreviation PO administration refers to per os.

As used herein, the abbreviation PI refers to polydispersity index.

As used herein, the abbreviation AUC refers to area under the curve.

As used herein, the abbreviation TGA refers to thermogravimetric analysis.

As used herein, the abbreviation ODT refers to oral disintegrating tablet.

As used herein, the abbreviation PPF-PNDS refers to propofol polymeric nanodelivery system.

As used herein, the abbreviation PEG refers to polyethylene glycol.

As used herein, the abbreviation CMC refers to critical micellar concentration.

As used herein, the abbreviation IV refers to intravenous.

### DEFINITIONS

The following section defines various terms and expressions used throughout the instant specification.

As used herein, the term “solid formulation” refers to a substantially dry, solid state, formulation prepared from drying (e.g. removing solvent from) a mixture of a liquid biologically active agent and at least one stabilizing agent in such a manner to form an intimate mixture of the liquid biologically active agent and the at least one stabilizing agent and, optionally, one or more additives.

As used herein, the term “stabilizing agent” refers to any vehicle or material which allows aqueous preparation of the liquid biologically active agent, which is capable of forming, under appropriate conditions, a nanodispersion or micelle loaded with the liquid biologically active agent, for example, an amphiphilic copolymer or surfactant.

As used herein, the term “liquid biologically active agent” refers to a hydrophobic or amphiphilic therapeutic agent that is liquid (e.g. oil), or can be liquefied, at temperatures between about 0° C. to about 100° C. Preferably, the liquid biologically active agent is liquid at room temperature, for example, between about 16° C. to about 25° C.

As used herein, the term “therapeutic agent” refers to an agent that has a therapeutic or health-promoting effect when administered to a human or an animal, for example, an agent capable of treating or preventing a disease or condition. Examples of therapeutic agents include, but are not limited to, drugs, prodrugs, vitamins and supplements.

As used herein, the term “additives” refers to excipients, carriers, diluents, and the like, having substantially no pharmacological activity. The additives are preferably “pharmaceutically acceptable” referring to additives which are nontoxic when administered to a patient in an amount sufficient to provide a therapeutic effect and which do not destroy the biological activity of the active agent.

As used herein, the term “hydrogel” refers to three-dimensional, water-swollen structures composed of mainly hydrophilic homopolymers or copolymers, for example, polycarbophilic acid. There are natural hydrogels and synthetic hydrogels. Typical examples of natural hydrogels are those comprising alginate or polysaccharides. Typical examples of synthetic hydrogels are those comprising polyvinyl alcohol (PVAL), polyvinyl pyrrolidone (PVP), polyethylene oxide (PEO), polyacrylamide (PAAm), polyacrylic acid (PM), or polyvinyl methyl ether (PVME). Hydroxypropyl distarch phosphates are another example of a hydrogel.

As used herein, the terms “intimate mixture” or “in intimate association with” means that at least a portion of the liquid biologically active agent is in intimate contact with the core (e.g. hydrophobic segment) of the stabilizing agent, for example, in the form of a nanodispersion or micelle loaded with the liquid biologically active agent.

As used herein, the term “nanodispersion” refers to a system of nanoparticles which are capable of sequestering a liquid biologically active agent. Examples include, for example, micelles, liposomes, nanocapsules, nanospheres, lipid complexes, cyclodextrin complexes, polymersomes, dendrimers, nanoemulsions, latexes and the like.

As used herein, the term “micelle” refers to a supramolecular self-assembly capable of sequestering a liquid biologically active agent, for example, to improve miscibility of the biological agent in an aqueous environment.

As used herein, the term “hydrophobic” means substantially immiscible with aqueous medium.

As used herein, the term “hydrophilic” means substantially miscible with aqueous medium.

As used herein, the term “amphiphilic” means having at least one hydrophobic segment and at least one hydrophilic segment.

As used herein, the term “hydration” refers to partial or full reconstitution of the solid formulation in an aqueous medium, for example, a biological fluid, water, or aqueous solution.

The term “powder” refers to a substantially dry, free-flowing, particulate material having high bulk density. Spray-dried powders typically have a bulk density in the range of about 0.05-1.00 g/cc, more typically between about 0.2-0.5 g/cc. Advantageously, powders are suitable for incorporation into various non-intravenous dosage forms, including but not limited to, tablets, including rapid disintegrating tablets, caplets, capsules, sachets, solutions, suspensions, creams, gels, ointments, pessaries, suppositories, enema, drops, aerosol or dry powder inhalers, and the like.

The term “cake”, as compared to a powder, refers to a non-flowing, non-particulate material having a low bulk density, typically in the range of about 0.0001-0.05 g/cc. In accordance with the methods disclosed herein, a cake may be formed, for example, as a result of lyophilization or freeze-drying.

As used herein, the term “substantially dry” indicates that the at least about 90%, preferably at least about 95%, 96%, 97%, 98%, 99%, or 99.9%, of the solvent has been removed during the drying process.

The expression, “under conditions to achieve nanodispersion or micelle formation” includes dissolving in one or more suitable solvents and, optionally, one or more of heating, cooling, pressurizing, mixing, shaking, stirring, vortexing, blending, homogenizing, sonicating, or the like.

As used herein, the term “dosage form” refers to a pharmaceutical composition comprising a solid formulation as described herein, together with one or more additives, in a form or device suitable for non-intravenous administration to a patient. Examples include, but are not limited to tablets, including rapid disintegrating tablets, caplets, capsules, sachet formulations, solutions, suspensions, emulsions, creams, gels, hydrogels, films, lozenges, chewing gum, pastes, ointments, sprays, aerosol inhalers, dry powder inhalers, suppositories, pessaries, enemas, and the like.

As used herein, the term “non-intravenous” or “non-intravenous administration” refers to any suitable route of administration other than by injection or infusion, in particular, it includes routes of administration involving contact with mucous membranes, such as oral, sublingual, intranasal, intrapulmonary, ocular, topical, rectal, urethral and vaginal. The route of administration may be “non-parenteral”, thereby excluding all forms of parenteral administration.

The term “enteral” refers to routes of administration involving the alimentary canal, digestive tract or intestinal which, as used herein, includes at least oral, sublingual, and rectal.

As used herein, the term “instant release” refers to a dosage form that releases the solid formulation within about 1 second to about 30 seconds. When the solid formulation is released in an aqueous environment, e.g. upon hydration, the solid formulation is capable of forming a nanodispersion or micelle loaded with the biologically active agent.

As used herein, the term “immediate release” refers to a dosage form that releases the solid formulation within about 30 seconds to about 45 minutes.

As used herein, the term “controlled release” refers to any of a number of dosage forms that are capable of controlling the release of the biologically active agent, for example, timed release, delayed release, sustained release, pH-dependent release, and so on.

As used herein, the term “sustained release” refers to a dosage form that releases the solid formulation within about 45 minutes to about 24 hours.

As used herein, the term “therapeutic efficacy” refers to achieving a desired therapeutic outcome in the treatment or prevention of a named disease or condition, such as, for example, efficacy in alleviating or eliminating symptoms either on a temporary or permanent basis, or preventing or slowing the appearance of symptoms of the named disease or condition.

As used herein, the term “treat” or “treating” means to alleviate or eliminate symptoms, either on a temporary or permanent basis, or to prevent or slow the appearance of symptoms of the named disease or condition. The act of treating may not eliminate symptoms altogether but will provide relief or improvement to the subject being treated.

As used herein, the term “disease or condition” refers to a disease, disorder, condition, pathology, or symptom of any of the foregoing.

The term “subject” is used interchangeably with “patient” herein and includes mammals, including humans and animals.

As used herein, the term “therapeutically effective amount” refers to an amount of the biologically active agent that, when administered to a patient, is sufficient to achieve a desired therapeutic efficacy. The therapeutically effective amount can vary depending, for example, on the active agent, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate therapeutically effective amount in any given instance may be ascertained by those skilled in the art or capable of determination by routine experimentation.

A “dose” refers to the amount of biologically active agent to be administered to a patient in a given unit(s) of a dosage form. The dose required to achieve therapeutic efficacy can vary depending on, for example, the disease or disorder to be treated, the dosage form, and the route of administration.

As used herein the term “AUC” is the area under a curve representing the concentration of a biologically active agent in a biological fluid of a patient within a defined period of time following administration of the biologically active agent to the patient. Examples of biological fluids include plasma, blood, lymphatic fluids and cerebro-spinal fluid. AUC may be determined by measuring the concentration of a biologically active agent in a biological fluid such as the plasma or blood over a given time period using known methods such as various chromatography methods and then calculating the area under the plasma concentration-versus-time curve. Suitable methods for calculating the AUC from a biologically active agent concentration-versus-time curve are well known in the art. As relevant to the disclosure herein, an AUC for propofol can be determined by measuring the concentration of propofol in a biological fluid of a patient following administration of a dosage form comprising propofol.

As used herein, “bioavailability” refers to the amount of a biologically active agent within a specific body compartment (such as the blood of the systemic circulation) of a patient, following administration of the biologically active agent to that patient, as a percentage of the amount of the biologically active agent administered. Bioavailability values may be expressed in terms of either absolute bioavailability or relative bioavailability. It is the absolute bioavailability of the biologically active agent in the body compartment that is of concern when comparing formulations developed for intravenous administration with those developed for non-intravenous administration.

Absolute bioavailability compares the bioavailability of the biologically active agent in the systemic circulation following non-intravenous administration (for example after oral, rectal, transdermal, subcutaneous, or sublingual administration), with the bioavailability of the same biologically active agent administered intravenously, that is; the AUC generated by the biologically active agent in the systemic circulation post non-intravenous administration compared with the corresponding AUC generated by intravenous administration of the same biologically active agent. The comparison must be dose normalized to account for different doses or varying weights of the subjects. Thus, the absolute bioavailability is the dose-corrected area under curve (AUC) for the non-intravenous dose divided by the AUC generated by the intravenous dose. For example, the formula for calculating the absolute bioavailability F for a biologically active agent administered by the oral route (po) is:

$$F = \frac{{[ AUC ]}_{po} * {dose}_{IV}}{{[ AUC ]}_{IV} * {dose}_{po}}$$

Therefore, a biologically active agent given by the intravenous route will have an absolute bioavailability of 1 (F=1) while biologically active agents given by other routes usually have an absolute bioavailability of less than one. Expressed as a percentage, a biologically active agent given by the intravenous route will have an absolute bioavailability of 100% while those administered by other routes will have values less than 100%.

As used herein, the term “apical side” refers to the surface of the plasma membrane of a polarized cell that faces the lumen.

As used herein, the term “basolateral side” refers to the surface of the plasma membrane of a polarized cell that forms its basal and lateral surfaces. It faces towards the interstitium, and away from the lumen.

When introducing elements disclosed herein, the articles “a”, “an”, “the”, and “said” are intended to mean that there are one or more of the elements. The terms “comprising”, “having”, “including” are intended to be open-ended and mean that there may be additional elements other than the listed elements.

As used herein, the term “about” in association with a numeric value or range refers to a variation of +/−10%.

Reference is now made in detail to embodiments of the present disclosure. The disclosed embodiments are not intended to be limiting of the claims. To the contrary, the claims are intended to cover alternatives, modifications, and equivalents.

Solid Formulations

It has now been found that a solid formulation of a liquid biologically active agent, as described herein, when administered in a non-intravenous dosage form, is capable of achieving sufficient plasma levels to have therapeutic effect in vivo. To date, such a solid formulation was only thought to be suitable for reconstitution and administration in an intravenous dosage form (see WO 2006/05064).

The present disclosure thus provides effective non-intravenous dosage forms suitable for use in a hospital or outpatient setting. Importantly, the present disclosure provides a means of converting a liquid biologically active agent, including some that are currently administered intravenously only, e.g. propofol, into a solid formulation suitable for administration in a non-intravenous dosage form. Moreover, the solid formulation, as described herein, when administered in a non-intravenous dosage form, may improve the bioavailability of a liquid biologically active agent compared to administration of the same agent alone.

The present disclosure thus provides a solid formulation of a liquid biologically active agent suitable for use in a non-intravenous dosage form.

The solid formulation comprises a liquid biologically active agent in intimate association with at least one stabilizing agent. The solid formulation, upon hydration, is capable of forming a nanodispersion or micelle loaded with the liquid biologically active agent.

The solid formulation may be obtained by drying (e.g. removing solvent or solvents from) a mixture of a liquid biologically active agent and at least one stabilizing agent and, optionally, one or more additives, in such a manner as to form an intimate mixture of the liquid biologically active agent and the stabilizing agent.

In some embodiments, the solid formulation is obtained by freeze-drying (e.g. lyophilizing) the mixture.

In some embodiments, the solid formulation is obtained by spray-drying the mixture or drying the mixture in a fluidized bed (e.g. fluid bed-drying). This method of obtaining the solid formulation poses additional challenge compared to freeze-drying, since the components of the mixture remain in the liquid state during the process, thereby providing opportunity for mixing of the liquid biological agent with the solvent, with potential for loss of active agent during the drying process. However, it has been found that, according to methods disclosed herein, substantially none of the active agent is lost.

Other suitable forms of drying known in the art may also be used.

In some embodiments, the formulation is in the form of a substantially dry powder or a cake.

A powder may be formed, for example, as a result of spray-drying or fluid bed-drying a mixture of a biologically active agent, at least one stabilizing agent, and a suitable solvent therefor. A cake may be formed, for example, as a lyophilizing or freeze-drying a mixture of a biologically active agent, at least one stabilizing agent, and a suitable solvent therefor.

In some embodiments, the powder is a spay-dried powder. In some embodiments, the powder is a fluid-bed dried powder. In some embodiments, the powder has a bulk density in the range of about 0.05-about 1.00 g/cc. In some embodiments, the powder has a bulk density in the range of about 0.2-about 0.5 g/cc. Advantageously, powders are suitable for incorporation into various non-intravenous dosage forms.

In some embodiments, the formulation is in the form of a “cake”. In some embodiments, the cake has a bulk density in the range of about 0.0001-about 0.05 g/cc.

The solid formulations are suitable for use in a number of different non-intravenous dosage forms, particularly, the powder formulations due to their free-flowing, particulate, nature as compared to cakes.

The solid formulation can improve the bioavailability of the biologically active agent compared to administration of the biologically active agent alone (e.g. in the absence of the stabilizing agent). For instance, as demonstrated in the Examples, oral administration of a reconstituted solid formulation comprising propofol increased propofol bioavailability levels compared to reported oral bioavailability levels of about 5-8%. The oral bioavailability levels of propofol demonstrated in the examples herein ranged from about 14% to about 165%, depending on the stabilizing agent used. Even a bioavailability of 14% represents an increase compared to reported values.

In some embodiments, the bioavailability of the active agent is at least about 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160% or higher. Bioavailability is typically measured as absolute bioavailability.

In one embodiment, the liquid biologically active agent is propofol and the bioavailability of propofol is at least about 10%, preferably between about 15% and about 165%, between about 15% and about 100%, between about 15% and about 80%, or between about 20% and about 80% compared to an equivalent intravenous dose of propofol.

In some embodiments, the bioavailability is improved or increased compared to same-route administration of the active agent in the absence of the stabilizing agent.

In some embodiments, the bioavailability of the active agent is increased by at least about 1.5-fold, 2-fold, 3-fold, 5-fold, 10-fold, 15-fold, 20-fold, 30-fold, 40-fold, 50-fold, 75-fold, 100-fold, 250-fold, 500-fold, 1000-fold, 1500-fold, 3000-fold, 5000-fold, 7500-fold, 10000-fold, or higher. In the examples provided herein, oral bioavailability of propofol was increased by about 3-fold to about 33-fold based on a reported oral bioavailability level of about 5%. Thus, in some embodiments, the bioavailability of the active agent is increased by at least about 1.5-fold to about 40-fold, from about 2-fold to about 35-fold, from about 5-fold to about 30-fold,

Solid formulations meeting desired criteria for a given application can easily be selected by those of skill in the art.

Liquid Biologically Active Agent

The liquid biologically active agent may be any liquid therapeutic agent that is compatible with the stabilizing agent used in the formation of the solid formulation, and which is capable of forming a nanodispersion or micelle loaded with the biologically active agent under appropriate conditions. It will be understood that the liquid biologically active agent is one which is compatible with the methods of preparation disclosed herein.

The biologically active agent comprises a hydrophobic or amphiphilic molecule, such as a hydrophobic or amphiphilic drug, prodrug, vitamin or supplement.

In some embodiments, the liquid biologically active agent is a liquid drug, such as, propofol, quinaldine, methoxyflurane, nicotine, phytonadione, methoxyflurane, dinoprost tromethamine, mesoprostol.

In some embodiments, the liquid biologically active agent is a liquid vitamin or supplement, such as alpha-linolenic acid, vitamin E, fish oil, an essential oil, or an extract.

In some embodiments, the liquid biologically active agent is present in the solid formulation in a therapeutically effective amount. The therapeutically effective amount can be determined by those of skill in the art.

In some embodiments, the therapeutically effective amount is an amount that, when administered to a subject, is capable of treating or preventing a disease or condition.

In some embodiments, the liquid biologically active agent is present in the solid formulation in an amount between about 1 wt % and about 80 wt %, 1 wt % and about 60 wt %, 5 wt % and about 40 wt %, between about 5 wt % and about 30 wt %, between about 10 wt % and about 30 wt %, between about 10 wt % and about 20 wt %, between about 0.1 wt % and 5 wt %, between about 1 wt % and about 5 wt %. This range can vary to a large extent as will be appreciated by one skilled in the art.

In some embodiments, the liquid biologically active agent is propofol or a derivative or prodrug thereof. Various prodrug forms of propofol are known from the prior art. A skilled person will be able to select those prodrug forms that are compatible with the present disclosure.

In some embodiments, the therapeutically effective amount is an amount of propofol that, when administered to a subject, is capable of treating or preventing a disease or condition. The disease or condition may, for example, be headache (e.g. migraine headache and/or intractable migraine headache), emesis and/or nausea (e.g. associated with chemotherapy or surgery), or pain (e.g. pain associated with cancer, central pain, surgical pain, neuropathic pain. For treatment of such conditions, propofol is preferably administered at a dose less than that required to achieve moderate sedation or anaesthesia.

wherein the neuropathic pain is chosen from post-herpetic neuralgia, peripheral neuropathy, trigeminal neuralgia, lower back pain, painful diabetic neuropathy, HIV-related neuropathic pain, cancer-related pain, and fibromyalgia.

In some embodiments, the therapeutically effective amount of propofol is an amount that, when administered to a subject, induces moderate sedation or anaesthesia.

Other known uses of propofol are also contemplated.

Although the liquid biologically active agent is referred to as a liquid, the skilled person will appreciate that, once incorporated into the dry solid formulation, it is no longer in true liquid form.

Micelles

The solid formulations described herein have the characteristic of forming micelles or nanodispersions upon hydration, for example, upon contact with an aqueous fluid, which may be an aqueous bodily fluid, such as saliva, mucous or gastric fluid. It has been found that the micelles form immediately and spontaneously upon hydration and will form across a wide range of pH levels, for example, from pH 1 to 12, depending on the stabilizing agent selected.

The micelles or nanodispersions allow high loading levels of propofol or other liquid biologically active agent to be achieved, with substantially no effect on stability. In some embodiments, the drug loading level (DLL) is up to about 5%, 10%, 15%, 20%, 25%, 50%, 60%, 70%, 80%, or higher. In some embodiments, the DLL is from about 1% to about 80%, from about 10% to about 80%, or from about 20% to about 60%.

Micelle formation occurs as a result of two forces. One is an attractive force that leads to the association of molecules, while the other is a repulsive force that prevents unlimited growth of the micelles to a distinct macroscopic phase. Amphiphilic copolymers self-associate when placed in a solvent that is selective for either the hydrophilic or hydrophobic polymer. The micellization process of amphiphilic copolymers is similar to that for low molecular weight surfactants. At very low concentrations, the polymers exist only as single chains. As the concentration increases to reach a critical value called the critical association concentration (“CAC”), polymer chains start to associate to form micelles in such a way that the hydrophobic part of the copolymer will avoid contact with the aqueous media in which the polymer is diluted.

Amphiphilic copolymers usually exhibit a CAC which is much lower than that of low molecular weight surfactants. For example, the CAC of PEO PBLA and PNIPA-PSt are between 5-20 mg/L. Some amphiphilic copolymers, however, exhibit much higher CAC, reaching up to 100 mg/L to 100,000 mg/L, as in the case of poloxamers. Amphiphilic copolymers with high CAC may not be suitable as drug targeting devices since they are unstable in an aqueous environment and are easily dissociated upon dilution. Preferred polymers are those having a relatively low CAC, for example, below about 1000 mg/L.

The micellization of amphiphilic copolymers can result in two different types of micelles depending on whether the hydrophobic chain is randomly bound to the hydrophilic polymer or grafted to one end of the hydrophilic chain. Micelles formed from randomly modified polymers are generally smaller than end-modified polymers. The micellar size is mainly determined by the hydrophobic forces which sequester the hydrophobic chains in the core, and by the excluded volume repulsion between the chains which limits their size. The difference in the balance of these two forces in random and end-modified copolymers may account for their different size.

Light scattering is widely used for the determination of the molecular weight and aggregation number of micelles.

A preferred method to determine the CAC involves the use of fluorescent probes, among which pyrene is widely used. Pyrene is a condensed aromatic hydrocarbon that is highly hydrophobic and sensitive to the polarity of the surrounding environment. Below the CAC, pyrene is solubilized in water, a medium of high polarity. When micelles are formed, pyrene partitions preferentially toward the hydrophobic domain afforded by the micellar core, and thus experiences a nonpolar environment. Consequently, numerous changes such as an increase in the fluorescence intensity, a change in the vibrational fine structure of the emission spectra, and a red shift of the (0,0) band in the excitation spectra are observed. The apparent CAC can be obtained from the plot of the fluorescence of pyrene, the 11/13 ratio from emission spectra or the 1338/1333 ratio from the excitation spectra versus concentration. A major change in the slope indicates the onset of micellization. Changes in anisotropy of fluorescent probes have also been associated with the onset 25 of micellization. E.g. see Jones and Leroux Eur. J. Pharm. Biopharm. I (1999) 48, 101-111.

In some embodiments, a “nanodispersion” is formed upon hydration of the solid formulation. In some embodiments, the nanodispersion comprises or consists of micelles, liposomes, nanocapsules, nanospheres, lipid complexes, cyclodextrin complexes, polymersomes, dendrimers, nanoemulsions, latexes or the like.

Polymeric micelles, such as those described herein, are characterized by their small size, typically less than about 500 nm. In some embodiments, the micelles formed are between about between about 5 nm to 500 nm, 10 nm to 500 nm, 10 nm to 400 nm, 20 nm to 300 nm, 20 nm to 200 nm.

Micellar size depends on several factors including copolymer molecular weight, relative proportion of hydrophilic and hydrophobic chains and aggregation number. Micellar diameter and size polydispersity can be obtained directly by dynamic light scattering (DLS) or other methods known to those skilled in the art.

Loading of one or more biologically active agents into the micelles can be realized according to techniques well known to one skilled in the art.

Stabilizing Agent

The stabilizing agent may be any material or vehicle capable of forming a nanodispersion or micelle loaded with the liquid biologically active agent under appropriate conditions.

In some embodiments, the stabilizing agent comprises at least one amphiphilic copolymer or at least one surfactant.

In some embodiments, the stabilizing agent comprises at least one amphiphilic copolymer. The amphiphilic copolymer may be a linear, branched or star-shaped polymer.

Suitable polymers are described herein below and also in, for example, WO 2006/056064, WO 02/100439, WO 03/077882, U.S. Pat. No. 6,939,564, WO 02/00194, WO 01/87227, U.S. Pat. No. 6,939,564, WO 02/100529, WO 03/078489, WO 2005/054319, WO 2007/073596, and WO 2008/035229.

Amphiphilic copolymers have at least one hydrophilic segment and at least one hydrophobic segment.

In some embodiments, the hydrophilic segment is selected from poly(ethylene oxide), poly(N-vinylpyrrolidone), poly(N-2-hydroxypropylmethacrylamide), poly(2-ethyl-2-oxazoline), poly(glycidol), poly(2-hydroxyethylmethacrylate), poly(vinylalcohol), polymethacrylic acid derivatives, poly(vinylpyridinium), poly((ammoniumalkyl)methacrylate), poly((aminoalkyl)methacrylate) and combinations and derivatives thereof; and a hydrophobic segment selected from the group comprising a poly(ester), poly(ortho ester), poly(amide), poly(esteramide) poly(anhydride), poly(propylene oxide), poly(tetrahydrofuran), polystyrene, polymethacrylate, polyacrylate, polymethacrylic acid, polyacrylic acid and combinations and derivatives thereof.

The hydrophobic segment may comprise a poly(ester) selected among poly(e-caprolactone), poly(lactide), poly(glycolide), poly(lactide-co-glycolide), poly(hydroxyl-alkanoates), poly(β-malic acid), and combinations and derivatives thereof.

In some embodiments, the amphiphilic copolymer comprises PVP-PDLLA or PEG-PMA. In some embodiments, the amphiphilic copolymer consists of PVP-PDLLA or PEG-PMA. Other amphiphilic copolymers, or combinations thereof, could also be used.

In some embodiments, the copolymer is a diblock or triblock copolymer.

In some embodiments, the amphiphilic copolymer is a PEG-PMA copolymer.

In some embodiments, the PEG-PMA copolymer is an EG-MAA-BMA block copolymer. Suitable EG-MAA-BMA block copolymers may, for example, have the following composition: EG₍₂₀₋₅₀₀₎-MAA₍₅₋₅₀₀₎-BMA₍₅₋₅₀₀₎. In some embodiments, the EG-MAA-BMA copolymer has the following composition: EG₍₃₅₋₅₀₎-MAA₍₅₀₋₇₀₎-BMA₍₂₀₋₄₀₎.

In one embodiment, the EG-MAA-BMA copolymer has one of the following structures: EG₍₄₅₎-MAA₍₆₃₎-BMA₍₂₈₎; EG₍₄₅₎-MAA₍₆₄₎-BMA₍₃₄₎; or EG₍₄₅₎-MAA₍₅₄₎-BMA₍₂₆₎.

In some embodiments, the copolymer is (PEG₄₅-b-P(MAA₅₀-co-nBMA₂₅)), PEG-b-P(DMAEMA₇₀-co-EMA₃₀); or PEG-b-P(EA₅₀-co-MAA₅₀).

In some embodiments, the amphiphilic copolymer is a PVP-PDLLA copolymer.

In one embodiment, the copolymer is a PVP-PDLLA copolymer having the following characteristics: % PDLLA: 34.4% (by TGA); M_{w}=4961; Mₙ=4177; PI=1.2 (P1).

In one embodiment, the copolymer is a PEG-PMA copolymer having the following characteristics: PEG-MAA-nBMA: 45-54-26; Mw=13600 (by SLS); Mn=10709 (by NMR); PI-1.28 (P3).

The stabilizing agent may also be a surfactant, such as lauryl sulphate, hexadecyl pyridinium chloride, polysorbates, sorbitans, poly(oxyethylene) alkyl ethers, poly(oxyethylene) alkyl esters and combinations thereof.

The stabilizing agent may further comprise a targeting moiety. Micelles presenting functional groups at their surface for conjugation with a targeting moiety have al been described in, for example, Scholz, C. et al., Macromolecules (1995) 28, 7295-7297).

In some embodiments, the CAC of the copolymers is in the range of about 0.1 mg/L to about 1000 mg/L, about 0.1 mg/L to about 100 mg/L, about 0.1 mg/L to about 50 mg/L, about 0.1 to about 25 mg/L, about 0.1 to about 10 mg/L, or about 0.1 to about 5 mg/L. Particularly preferred polymers have a low CAC, for example, below 100 mg/L, below about 50 mg/L, below about 25 mg/L, below about 10 mg/L, or below about 5 mg/L. CAC may be determined, for example, by measuring the influence of polymer concentration on the excitation shift of pyrene fluorescence on a Varian fluorimeter.

Without being bound to the theory, it is believed that the formulation described herein, when administered to a mammal, is capable of producing loaded micelles or nanodispersions which result in a sufficient bioavailability for the purpose of medical use, for example, for achieving therapeutic efficacy.

Dosage Forms

The solid formulation can be formulated in a dosage form suitable for non-intravenous administration, for example, a dosage form for oral, sublingual, intranasal, intrapulmonary, rectal, urethral, vaginal, ocular, otic, or topical administration. Such dosage forms are generally suitable for use in either a hospital or outpatient setting.

In some embodiments, the dosage form is for enteral administration.

In one embodiment, the dosage form is for oral administration.

In one embodiment, the dosage form is for sublingual administration.

In some embodiments, the dosage form is selected form the group consisting of pills, tablets, caplets, capsules, sachet formulations, solutions, suspensions, emulsions, creams, gels, films, lozenges, chewing gum, pastes, drops, ointments, sprays, aerosol inhalers, dry powder inhalers, suppositories, pessaries, enemas, and the like.

In one embodiment, the dosage form is a rapid disintegrating tablet. A rapid disintegrating tablet is one which comprises a disintegrant or disintegrating matrix to facilitate rapid release of the solid formulation from the dosage form. In some embodiments the disintegrating matrix is provided by a starch or a hydrogel. In some embodiments, the starch is a cross-linked high amylose starch, such as Contramid™ (Labopharm Inc, Quebec, CA).

In some embodiments, the solid formulation is present in the dosage form in an amount from about 1 wt % to about 99 wt %, from about 5 wt % to about 85 wt %, from about 5 wt % to about 60 wt %, 5 wt % to about 40 wt %, between about 5 wt % to about 30 wt %, between about 10 wt % to about 30 wt %, between about 10 wt % to about 20 wt %, between about 0.1% to 5%, between about 1 wt % to about 5 wt %, between about 20 wt % to about 60 wt %. This range can vary to a large extent as will be appreciated by one skilled in the art.

In some embodiments, the biologically active agent is present in the dosage form in an amount from about 0.01 wt % to about 80 wt %, 0.01 wt % to about 50 wt %, from about 1 wt % to about 20%, from about 1 wt % to about 15 wt %, from between about 2 wt % to about 10 wt %, between about 1 wt % to about 5 wt %, between about 5 wt % to about 10 wt %, or between about 10 wt % to about 20 wt %. This range can vary to a large extent as will be appreciated by one skilled in the art.

Additives

The dosage form may consist of the solid formulation in a suitable vehicle, such as a capsule or sachet. Optionally, the dosage form may comprise the solid formulation and one or more additives. The additives are preferably pharmaceutical grade and may include, for example, a carrier, a bulk forming agent, a cryoprotectant, a lyoprotectant, a binder, a flavoring agent, a taste masking agent, a coloring agent, an odorant, a buffer, a preservative, a diluent, a dispersant, a surfactant, a disintegrant, or an additional stablilizer.

In some embodiments, tablet is a rapid disintegrating tablet (RDT) comprising a disintegrant or disintegrating matrix to facilitate rapid release of the biologically active agent from the dosage form. In some embodiments, the disintegrating matrix is a starch or a hydrogel. In some embodiments, the starch is a cross-linked high amylose starch. In some embodiments, the additive is a cross-linked starch, such as a cross-linked high amylose starch. In some embodiments, the cross-linked high amylose starch is Contramid® (Labopharm, Quebec, CA) In some embodiments, the RDT additionally comprises a sugar, such as, mannitol, trehalose, maltodextran.

Other suitable additives include, but are not limited to poly(vinylpyrrolidone), poly(ethylene glycol), sugars (lactose, trehalose), polyols (mannitol), saccharides and amino acids.

Flavouring agents may, for example, include a sweetener, such as an artificial sweetener. The artificial sweetener may be, for example, aspartame or sucralose.

A bulk forming agent may, for example, be a commercially available poly(vinylpyrrolidone), such as, Kollidon® 12 PF or 17 PF (BASF).

In the case of tablets, carriers that are commonly used include lactose, sodium citrate and salts of phosphoric acid. Various disintegrants such as starch, and lubricating agents such as magnesium stearate and talc, are also commonly used in tablets.

For oral administration in capsule form, useful diluents are lactose and high molecular weight polyethylene glycols. If desired, certain sweetening and/or flavoring agents are added.

For ocular administration, ointments or droppable liquids may be delivered by delivery systems known to the art such as applicators or droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents and/or carriers. They may also include buffers and antioxidants.

For pulmonary administration, diluents and/or carriers will be selected to be appropriate to allow the formation of an aerosol or dry powder inhaler.

Suppository dosage forms are useful for vaginal, urethral and rectal administrations. Such suppositories will generally be constructed of a mixture of substances that is solid at room temperature but melts at body temperature. The substances commonly used to create such vehicles include the obroma oil, glycerinated gelatin, hydrogenated vegetable oils, mixtures of polyethylene glycols of various molecular weight and fatty acid esters of polyethylene glycol. See, Remington's Pharmaceutical Sciences, 16th Ed., Mack Publishing, Easton, Pa., 1980, pp. 1530-1533 for further discussion of suppository dosage forms.

Gels, creams, ointments and pastes can be used for vaginal, urethral and rectal and topical administrations.

In some embodiments, the dosage form is one which will enhance delivery of the biologically active agent to the brain, such as a sublingual disintegrating tablet or nasal or pulmonary inhaler.

The dosage form may be an instant release dosage form, an immediate release dosage form, or a controlled release dosage form. In some embodiments, the dosage form is a controlled release dosage form and the controlled release is sustained release, for example, wherein the dosage form releases the liquid biologically active agent over a period of about 45 minutes to about 24 hours. In some embodiments, the dosage form releases the liquid biologically active agent over a period of at least about 4 hours, at least about 8 hours, at least about 12 hours, at least about 16 hours, at least about 20 hours, or at least about 24 hours.

Dosage forms may have instant release, immediate release or controlled release characteristics. Immediate release oral dosage forms release the propofol from the dosage form within about 30 minutes following ingestion. In certain embodiments, an oral dosage form provided by the present disclosure may be a controlled release dosage form. Controlled delivery technologies may improve the absorption of a drug in a particular region or regions of the gastrointestinal tract. Controlled drug delivery systems may be designed to deliver a drug in such a way that the drug level is maintained within a therapeutically effective blood concentration range for a period as long as the system continues to deliver the drug at a particular rate. Controlled drug delivery may produce substantially constant blood levels of a drug as compared to fluctuations observed with immediate release dosage forms. For some diseases maintaining a controlled concentration of propofol in the blood or in a tissue throughout the course of therapy is desirable. Immediate release dosage forms may cause blood levels to peak above the level required to elicit the desired response, which may cause or exacerbate side effects. Controlled drug delivery may result in optimum therapy, reduce the frequency of dosing, and reduce the occurrence, frequency, and/or severity of side effects. Examples of controlled release dosage forms include dissolution controlled systems, diffusion controlled systems, ion exchange resins, osmotically controlled systems, erodible matrix systems, pH independent formulations, gastric retention systems, and the like.

Controlled release oral dosage forms may additionally include an exterior coating to provide, for example, acid protection, ease of swallowing, flavor, identification, and the like.

Various controlled release preparations are described, for example, in WO 2004/038428, WO 2010/028489, WO 02/02084, WO 94/02121, WO 98/35992, WO 99/43305. Controlled release tablets capable of being bisected while maintaining substantially the same release profile of active agent are described, for example, in WO 2007/048219. Misuse preventative formulation are described, for example, in WO 2009/076764 and WO 2010/069050.

Regardless of the specific dosage form used, propofol may be released from the administered dosage form over a sufficient period of time to provide prolonged therapeutic concentrations of propofol in blood of a patient. Following administration, dosage forms comprising propofol may provide a therapeutically effective concentration of propofol in the blood of a patient for a continuous time period of at least about 4 hours, of at least about 8 hours, for at least about 12 hours, for at least about 16 hours, and in certain embodiments, for at least about 20 hours following administration of the dosage form to the patient. The continuous period of time during which a therapeutically effective blood concentration of propofol is maintained may begin shortly after oral administration or following a time interval.

For administration by intranasal or intrapulmonary inhalation or insufflation, the formulation may be formulated into an aqueous or partially aqueous solution, which can then be utilized in the form of an aerosol. Dry powder inhalers may also be used.

Methods

In one aspect, there is provided a method for the preparation of a solid formulation as defined herein which comprises forming a first mixture comprising a solution of at least one stabilizing agent and at least one solvent, under conditions to achieve micelle or nanodispersion formation, adding at least one liquid biologically active agent to said first mixture in a manner to load said micelle or nanodispersion therewith and form a second mixture, treating said second mixture to remove said solvent therefrom, while forming a substantially solid product that contains said liquid biologically active agent intimately associated with said stabilizing agent, said solid product upon hydration being capable of forming a nanodispersion or micelle loaded with said at least one biologically active agent.

In another aspect, there is provided a method for the preparation of a dosage form for non-intravenous administration of a liquid biologically active agent which comprises: providing a first mixture of at least one stabilizing agent in at least one solvent, under conditions to achieve micelle or nanodispersion formation, providing a second mixture by mixing said first mixture and at least one liquid biologically active agent to load said micelle or nanodispersion with said liquid biologically active agent, removing the solvent from said second mixture to form a solid formulation; and optionally, adding one or more additives suitable to prepare the non-intravenous dosage form.

In some embodiments, the solvent is removed by drying. In some embodiments, the drying involves spray drying or drying in a fluid bed. In some embodiments, the drying freeze drying.

In some embodiments, the biologically active agent may be pre-treated before being mixed with the stabilizing agent, for example, by heating or cooling to achieve a suitable liquid state.

The solid formulations according to the present invention can be prepared for example by any of the procedures disclosed in copending U.S. application Ser. No. 11/286,301 filed Nov. 25, 2005 and U.S. Pat. No. 6,939,564, which are incorporated herein by reference, in their entirety.

The method relies on a treatment, such as lyophilization, spray drying, fluid bed drying or the like well known to those skilled in the art, which is obtained by mixing a solvent selected from water, or an aqueous solution, or non-aqueous solvent, or combinations thereof with at least one stabilizing agent under conditions to provide a first solution, to which is added at least one liquid biologically active agent, such as propofol or the like, to give a second solution. The latter is lyophilized, spray-dried, subjected to solvent removal in a fluid bed, or the like, under conditions which yield a solid product, in which the liquid biologically active agent is intimately associated, and from which substantially all the solvent or solvents have been removed. Preferably, the solvent removal process results in virtually no loss of drug during the treatment. Optionally, one or more additives may be added at any stage during the treatment.

While in liquid state, the mixture could be subjected to a sterilizing filtration step prior to the above treatment which involves drying to form a powder, a cake or the like. The solid product resulting from the above treatment is a material that can be stored, easily transported and incorporated into dosage forms for non-intravenous administration, such as oral, sublingual, intranasal, intrapulmonary, rectal, urethral, vaginal, ocular, otic or topical route. Optionally, the solid formulation may be dispersed in a liquid dosage form, such as a solution, suspension, syrup elixir, or drop, for non-intravenous administration

The instant process illustrates a simple and elegant procedure for forming a solid formulation from a liquid containing an intimate association of an insoluble liquid drug and a stabilizing agent. The liquid, comprising an intimate association of the solvent, insoluble liquid drug and stabilizing agent, may be dried by a process, whereby the insoluble liquid drug remains in close association with the stabilizing agent such that virtually all drug is retained during the process. The product is a substantially dry solid as mentioned above. The dry solid product, upon hydration, spontaneously forms a nanodispersion or micelle or loaded with a liquid biologically active agent.

Suitable solvents or mixtures thereof will have the ability to solublize appropriate amounts of the stabilizing agent without denaturation or degradation of the liquid biological agent. Suitable solvents (or mixtures of solvents) are those capable of being removed during the drying process, e.g. lyophilization, spray-drying, fluid bed, or the like process. While numerous solvents are capable of functioning in accordance with the process disclosed herein, non-limiting illustrative examples of such solvents include water, aqueous solutions which may be pH adjusted, dextrose solution in water, saline, DMSO, DMF, dioxane, pyridine, pyrimidine, and piperidine, alcohols such as methanol, ethanol, n-butanol and t-butanol, and acetone, which are useful either alone or in combination, and may be further admixed, e.g. with water, to form a binary mixture. Other solvents may be added in small amounts to facilitate the dissolution of the drug.

In accordance with some embodiments, a predetermined amount of a stabilizing agent, e.g. a suitable polymer, copolymer or a surfactant, and optionally, an additive, e.g. a buffer, a cryoprotectant, a lyoprotectant, a bulk forming agent or the like, and/or additional stabilizing agents are dissolved in a solvent, e.g. water, an aqueous solution, at least one non-aqueous organic solvent, or combinations of water or an aqueous solution and said at least one non-aqueous organic solvent to form a first mixture in the form of a micellar solution. It has been realized that proper mixing can aid in achieving micelle or nanodispersion formation within the first mixture.

Once the first mixture is well formed, a liquid drug, here propofol, although any other liquid biologically active agent may be used as will be appreciated by one skilled in the art, is added to the first mixture under conditions well known to those skilled in the art, whereby the micelle or nanodispersion will be loaded with the liquid drug in a second mixture in the form of a drug micellar clear solution.

In either or both of the mixing steps described above, a suitable “additive” could be added for purposes well known to those skilled in the art. Non limiting examples of additives include, but are not limited to buffers, cryoprotectants, lyoprotectants and bulk forming agents. Other suitable additives include, but are not limited to poly(vinylpyrrolidone), polyethylene glycol), sugars (lactose, trehalose), polyols (mannitol), saccharides and amino acids soluble in the solvent or solvent mixture. As broadly recited herein, the term “solvent” is understood to mean water alone, water with at least one non-aqueous organic solvent, or combinations of water and said at least one non-aqueous organic solvent.

In one illustrative embodiment, additional dissolution enhancing means, here stirring, may be employed to aid in the forming of the liquid comprising a biologically active agent, a stabilizing agent and a solvent, prior to treatment to form a solid product. Illustrative, but non-limiting examples of said dissolution enhancing means may include a process, for example, wherein the mixture may be stirred, vortexed, or sonicated, if needed. For some polymers, the solution may also need to be heated to speed up dissolution.

Optionally, the solution may filtered through a sterilizing filter, e.g. through a 0.2 μm filter. Subsequently, the solution is freeze-dried to form a sterile dry cake or powder or the like.

The solid formulation may be first formed and then subsequently incorporated into a dosage form suitable form non-intravenous administration. Alternatively, the components of the solid dosage form may be combined with additional additives required to make the non-intravenous dosage form and the resulting mixture may be dried to form the dosage form comprising the solid formulation.

Methods of Treatment

In another aspect, the present disclosure provides a method or treating a disease or condition, comprising administering to a subject in need thereof, typically a mammal selected from a human or animal, a therapeutically effective amount of a non-intravenous dosage form as described herein.

The dosage form may be administered by any suitable non-intravenous route as may be determined by a skilled professional. In some embodiments, the route of administration is oral, sublingual, intranasal, intrapulmonary, rectal, urethral, vaginal, ocular. otic or topical administration. In some embodiments, the dosage form is for enteral administration.

In one embodiment, the route of administration is oral administration.

In one embodiment, the route of administration is sublingual administration.

In some embodiments, the disorder or condition to be treated is a disorder or condition of the central nervous system, such as, headache, nausea, emesis or pain.

In some embodiments, the headache is migraine headache, such as intractable migraine headache. In some embodiments, the emesis or nausea is due to cancer chemotherapy or surgery. In some embodiments, the pain is cancer pain, central pain, neurophathic pain or surgical pain. In some embodiments, the neuropathic pain is post-herpetic neuralgia, peripheral neuropathy, trigeminal neuralgia, lower back pain, painful diabetic neuropathy, HIV-related neuropathic pain, cancer-related pain, and fibromyalgia

In some embodiments, there is provided a method of treating or preventing headache, nausea, emesis or pain, comprising administering to a subject in need thereof a therapeutically effective amount of a dosage form as described herein which comprises propofol as an active ingredient. A subject in need thereof is a subject suffering from, prone to, or anticipated to suffer from, one or more of headache, nausea, emesis or pain.

In some embodiments, there is provided a method of treating or preventing intractable migraine headache.

In another aspect, there is provided a method of treating or preventing headache, nausea, emesis or pain, comprising administering to a subject in need thereof a therapeutically effective amount of a non-intravenous dosage form comprising a solid formulation, and, optionally, one or more additives, the solid formulation comprising an intimate mixture of propofol and at least one amphiphilic copolymer, wherein, upon hydration, micelles loaded with the propofol are formed.

The dosage form may be administered in a suitable amount to achieve therapeutic efficacy without significant toxicity or side effects. In some embodiments, the dosage form is administered in an amount sufficient to achieve a therapeutically effective amount of the biologically active agent in the blood or plasma of a subject treated with the dosage form.

The dosage requirements vary with the particular formulations and dosage forms employed, the route of administration, the severity of the symptoms presented and the particular subject being treated. Treatment will generally be initiated with small dosages less than the optimum dose of the compound. Thereafter the dosage is increased until the optimum effect under the circumstances is reached. Precise dosages for, rectal, urethral, vaginal, ocular or topical administration will be determined by the administering physician based on experience with the individual subject treated. In general, the active agent is most desirably administered at a concentration that will generally afford effective results without causing harmful or deleterious side effects, and can be administered either as a single unit dose, or if desired, the dosage may be divided into convenient subunits at suitable times throughout the day.

In addition, in vitro or in vivo assays may optionally be employed to help identify optimal dosage ranges. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

The amount of a active agent administered can depend on, among other factors, the patient being treated, the weight of the patient, the health of the patient, the disease being treated, the severity of the affliction, the route of administration, the potency of the compound, and the judgment of the prescribing physician.

The amount of active agent that will be effective in the treatment of a particular disease, disorder, or condition disclosed herein will depend on the nature of the disease, disorder, or condition, and can be determined by standard clinical techniques known in the art.

A dose may be administered in a single dosage form or in multiple dosage forms. When multiple dosage forms are used the amount of active agent contained within each of the multiple dosage forms may be the same or different.

In certain embodiments, an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans.

With respect to propofol, a dose of a highly bioavailable agent may be within a range of circulating concentrations in for example the blood, plasma, or central nervous system, that is therapeutically effective, that is less than a sedative dose, and that exhibits little or no toxicity. A dose may vary within this range depending upon the dosage form employed.

During treatment a dose and dosing schedule may provide sufficient or steady state systemic concentrations of a therapeutically effective amount of propofol to treat a disease. In certain embodiments, an escalating dose may be administered.

The active agent may be administered at intervals for as long as necessary to obtain an intended or desired therapeutic effect.

Uses

The solid formulations and dosage forms described herein may be used in a number of different therapeutic applications. Thus, another aspect of the disclosure includes uses of the solid formulations and dosage forms described herein.

In one embodiment, there is provided a use of a non-intravenous dosage form as described herein in the manufacture of a medicament. In one embodiment, there is provided a non-intravenous dosage form as described herein for use in the manufacture of a medicament.

In one embodiment, there is provided a use of a solid formulation as described herein in the manufacture of a non-intravenous dosage form for treating or preventing a disease or condition. In one embodiment, there is provided a solid formulation as described herein for use of in the manufacture of a non-intravenous dosage form for treating or preventing a disease or condition.

In some embodiments, the biologically active agent is propofol. Thus, in one embodiment, there is provided a use of a solid formulation comprising an intimate mixture of propofol and at least one stabilizing agent, in the manufacture of a non-intravenous dosage form for the treatment or prevention of a disease or condition of the central nervous system. In another aspect, there is provided a use of a dosage form as described herein for the treatment or prevention of a disease or condition of the central nervous system. In some embodiments, condition of the central nervous system is headache, emesis, nausea, or pain.

In another embodiment, there is provided a solid formulation comprising an intimate mixture of propofol and at least one stabilizing agent, for use in the manufacture of a non-intravenous dosage form for the treatment or prevention of headache, nausea, emesis, or pain.

In some embodiments, the dosage form is for inducing anaesthesia or sedation in a subject in need thereof. In some embodiments, the dosage form is for use in the manufacture of a medicament for inducing anaesthesia or sedation in a subject in need thereof.

In another aspect, there is provided a use of a solid formulation comprising an intimate mixture of propofol and at least one amphiphilic copolymer in the manufacture of a non-intravenous dosage form for the treatment or prevention of a disease or condition of the central nervous system.

In another aspect, there is provided a solid formulation comprising an intimate mixture of propofol and at least one stabilizing agent, for use in the manufacture of a non-intravenous dosage form for the treatment or prevention of headache, nausea, emesis, or pain.

Kits and Commercial Packages

In another aspect of the disclosure, there are provides commercial packages and kits comprising a non-intravenous dosage form as described herein, together with one or more instructions for use in the treatment or prevention of a disease or condition.

The dosage form and, optionally, other components of the kit or commercial package, may be packaged in an appropriate container and, associated with such containers, can be a notice in the form prescribed by a governmental agency regulating the manufacture, use or sale of pharmaceutical or biological products, which notice reflects approval by the agency of manufacture, use or sale for human or animal administration.

When the components of the kit or commercial package may be provided in one or more liquid solutions, the liquid solution can be an aqueous solution or suspension, for example, a sterile aqueous solution or suspension. In this case the container means may itself be an inhaler, syringe, pipette, eye dropper, nasal dropper, ear dropper, or other such like apparatus, from which the formulation may be administered to a patient. The container may also be a dry powder inhaler.

Irrespective of the number or type of containers, the kit or commercial package may comprise an instrument for assisting with the administration of the composition to a patient. Such an instrument may be an inhalant, syringe, pipette, forceps, measured spoon, eye dropper or any such medically approved delivery vehicle.

The following examples are provided to assist the reader. The examples are not intended to limit the scope of the disclosure. While the liquid biologically active agent exemplified is propofol, it is understood that other liquid biologically active agents could also be used with similar results as will be appreciated by one skilled in the art.

With respect to polymer formulae, the subscript text indicates the repeat number in a polymeric segment. The letter b features that polymers and/or polymeric arms are based on a diblock copolymeric structure. The term co means the repeating units are disposed randomly along the polymeric segment.

### EXAMPLES

### Example 1

### Synthesis of diblock PEG-PMA

80 g poly(ethylene glycol) (MW 2,000, 40.0 mmol) is dried by azeotropic distillation with 250 ml toluene (bath set at 140° C.). After the polymer is cooled down to room temperature, 2400 mg KH (60.00 mmol, 4000 mg (4.0 ml) 30% KH dispersion in mineral oil) is added under argon atmosphere. 850 ml freshly distilled THF is added to dissolve the polymer. The reaction between KH and PEG is carried out for 120 min under rigid stirring. Then, without any distillation 256 mL of t-BMA (d 0.875, 224 g MW142.2 and 1575.2 mmol) and 130 mL of n-BMA (d 0.894, 116.2 g MW142.2, 817.3 mmol) are added to the reaction mixture and the solution is stirred for a further 120 min at 20° C. for the block copolymerization to take place. The polymer was collected by evaporation of solvent. Without any purification and characterization, the crude polymer was hydrolyzed with 320 mL of concentrated HCl in dioxane. The mixture is refluxed overnight. A PEG-PMA with the following empirical structure is obtained: (PEG₄₅-b-P(MMA₅₀-co-nBMA₂₅))

The resulting product is concentrated to about 600 mL by rotovap. 600 mL of water are added to the concentrated solution under rigid stirring. 100 mL of the new solution are retrieved and are added to a dialysis membrane (30 cm is required, molecular weight cut off (MWCO) of 3500, internal diameter 47 nm) and the dialysis membrane is put into distilled water (5 membranes are used per 5 L of water). Water is changed as frequently as possible, especially at the beginning, until the obtained pH is between 6 and 7. Each solution is transferred to a cake plate and frozen at −80° C. overnight. If desired, the solutions may be freeze-dried. The obtained product is a white powder.

### Example 2

### Synthesis of Triblock PEG-PMA (P4 and P5)

60.05 g of poly(ethylene glycol) methyl ether (PEGMe) (M_{w}, 2,000, 30.0 mmol) are dried under vacuum while stirring at 110° C. for 16 hours. After the polymer is cooled down to room temperature, 900 mL of freshly distilled THF are added. When the polymer is completely dissolved and the solution is at room temperature, 1800 mg KH (45.00 mmol, 6000 mg (6.0 ml) 30% KH dispersion in mineral oil) are added under an atmosphere of argon. The reaction between KH and PEGMe is carried out for 120 min under rigid stirring. Then, 192 mL of t-BMA (d 0.875, 168 g M_{w} 142.2, 1.181 mol) is added to the reaction vessel. The solution is stirred for a further 120 min at 20° C. for the diblock copolymerization to take place. Once reaction with t-BMA is completed, 108 mL of n-BMA (d 0.894, 96.55 g M_{w} 142.2, 678.98 mmol) are added to the reaction vessel using an addition flask. The solution is stirred for a further 120 min at 20° C. for the triblock copolymerization to take place. When the reaction is completed, the polymer was collected by evaporation of solvent. Without any purification and characterization, the crude polymer was hydrolyzed with 320 ml of concentrated HCl [>1.5 eq of HCl (≈3.75 mol HCl 320 mL HCl_{conc})] and 780 mL of dioxane are added to the polymer solution. This new mixture is kept under reflux overnight at 110° C. After the hydrolysis was completed, the solution was concentrated to about 600 mL, and the polymer was precipitated in cold water (ca. 2000 mL). The polymer was then centrifuged at 10000 rpm for 10 min. To remove the remaining impurities, the crude polymer obtained in previous step is dissolved in THF (as little as possible) and precipitated again in cold water (ca. 2000 mL). The polymer was then centrifuged at 10000 rpm for 10 min.

Dissolve again the obtained polymer in THF and repeat the precipitation and the centrifuge processes. Dry the final polymer.

Characterization of the PEG-PMA diblock (P3) and triblocks (P4 and P5) was performed by different techniques. The composition of the block copolymer and their molecular weight (Mₙ) was assessed by ¹H NMR and the critical micellar concentration (CMC) was determined by measuring the influence of polymer concentration on the excitation shift of pyrene fluorescence on a Varian fluorimeter (M. Francis et al. J. Control. Release, 93:59 (2003)). Acid content and pKa were determined by titration using an auto titrator (Malvern), molecular weight (M_{w}) was determined by light scattering (Malvern Zetasizer). Characteristics are presented in Table 1.

TABLE 1
Physicochemical characteristics of PEG-PMA triblock
polymers (P4 and P5) vs diblock copolymer (P3)

| | | Molecular | | | | | | | |
|---|---|---|---|---|---|---|---|---|---|
| | Structure | weight (KDa) | | CMC (mg/L) | | | | | |
| Poly- | (EG-MAA- | Mₙ | M_{w} | pH | pH | pH | pH | Acid | |
| mers | BMA) | (NMR) | (LS) | 5 | 6 | 7 | 10 | content | pKa |
| P3 | 45-63-28 | 11.5 | 14.1 | 2.3 | 2.0 | 1.6 | 6.9 | 30.6% | 6.7 |
| P4 | 45-64-34 | 12.3 | 21.2 | 0.6 | 0.7 | 0.5 | 0.7 | 29.6% | 6.3 |
| P5 | 45-54-26 | 10.3 | 22.5 | 1.2 | 0.7 | 1.2 | 1.2 | 25.9% | 6.0 |

### Example 3

### Preparation of PEG-PMA Formulation by Spray-Drying

A 50 mg/mL solution of PEG-PMA is prepared in 0.1 N NaOH. Sonication is used in order to get a complete dissolution of the polymer. An appropriate amount of solid NaOH is added until obtaining a final pH=8. Propofol is added to the polymer solution in order to get desired drug loading level (weight ratio drug/(polymer+drug)) (eg. 10% w/w) under vigorous magnetic stirring. The solution is stirred overnight. Deionized water is added in a quantity to obtain a final concentration of 5 mg/mL PPF.

The formulation was spray-dried using a lab-scale spray drier Buchi B-290 with the following conditions:

| | Main air | Spray | |
|---|---|---|---|
| Spray | flow | rate | Temperature (° C.) |

| nozzle | (m³/h) | (mL/min) | Inlet | product |
|---|---|---|---|---|
| 1.5 mm | 40 | 7 | 170 | 80 |

The yield of the spray drier powder is 86%.

The characteristics of the formulation are shown in Table 2.

TABLE 2
Spray-dried formulation characteristics as in example 3

| | | Drug | | Average |
|---|---|---|---|---|
| | | Loading level | | micelle |
| Formulation | Polymer | (% w/w)* | Assay | size (nm) |
| PM4 SD | P4 | 10 | 77% | 477 |
| PM5 SD | P5 | 10 | 63% | 216 |

### Example 4

### Preparation of PEG-PMA Formulation Using a Fluid-Bed Dryer

In this example, 20 mL of aqueous solution of PEG-PMA micelles loaded with 10% of propofol was sprayed on 50 g fast-flow lactose using the Huttlin fluid bed equipment. Experimental conditions for this trial are summarized in table 3.

TABLE 3
Experimental conditions of lactose-PEG-PMA granulation

| | Main air | Pressure | Spray | | |
|---|---|---|---|---|---|
| Spray | flow | spray | rate | Temperature (° C.) | |
| nozzle | (m³/h) | (bar) | (g/min) | Inlet | product |
| 0.8 mm | 10 | 0.5 | 0.4 | 40 | 27 |

The dry granules were then solubilised in deionised water and micelle-size was measured. Also propofol content was determined by HPLC method.

The results are shown in Table 4 below:

TABLE 4
Assay and micelle-size of granulated lactose-PEG-PMA-propofol

| | Average micelle-size | Assay |
|---|---|---|
| | (nm) | (%) |
| Before granulation | 136 | 100% |
| After granulation | 66 | 73% |

### Example 5

### Preparation of PEG-PMA Formulation by Freeze-Drying

A 50 mg/mL solution of PEG-PMA in 0.1 N NaOH is prepared. Sonication was used in order to get complete dissolution of the polymer. A drug solution is added to the polymer solution in order to get desired drug loading level (weight ratio drug/(polymer+drug)) under vigorous magnetic stirring, e.g. 10% w/w. The solution is stirred overnight. Deionized water is added to obtain a final concentration of 5 mg/mL PPF. The solution is divided into aliquots and each formulation is freeze-dried.

The cakes obtained were white and none of them melted. The protocol for the preparation of the formulation is summarized below in Table 5.

Characteristics of the above PPF formulations are given in the following Table 5.

TABLE 5
Freeze-dried formulation characteristics as example 5

| | | Drug | | | Visual |
|---|---|---|---|---|---|
| | | Loading | | Average | Stability |
| Formu- | | level | | micelle | Precipitation |
| lation | Polymer | (% w/w)* | Assay | size (nm) | time |
| PM3 FD | P3 | 10 | 52% | 150 | 24 h |
| PM5 FD | P5 | 10 | 92% | 278 | 24 h |

Similar propofol products can be prepared using polymers of the same nature and by using the above procedures or the procedures disclosed in U.S. Ser. No. 11/286,301.

Legends of propofol formulations used in the in vitro and in vivo studies are given in table 6.

TABLE 6
Propofol Formulations

| Abbreviation | DLL %* | Description | Polymer |
|---|---|---|---|
| PM1a | 10 | PVP-PDLLA | P1 |
| PM1b | 20 | | |
| PM1c | 10 | | |
| PM2 | 10 | PVP-PDLLA | P2 |
| PM3 FD | 10 | PEG-PMA | P3 |
| PM3 SD | 10 | | |
| PM4 FD | 10 | PEG-PMA | P4 |
| PM4 SD | 10 | | |
| PM5 FD | 10 | PEG-PMA | P5 |
| PM5 SD | 10 | | |

*Drug loading level is calculated from the amount of drug and polymer used during the formulation process: DLL % = 100% × (amount of drug/(amount of drug + amount of polymer))

### Example 6

### In Vitro Permeability Studies

Permeability studies were performed in vitro in a well established model of drug bioavailability. Caco-2 cells were seeded onto 12-well polyester filter membranes at a cell density of 60,000 cells/filter and cultivated for 21 days. Transport of PPF from Apical to Basolateral sides was evaluated after 120 min at 37° C. Formulations were dissolved in Hank's buffer media pH 6.8.

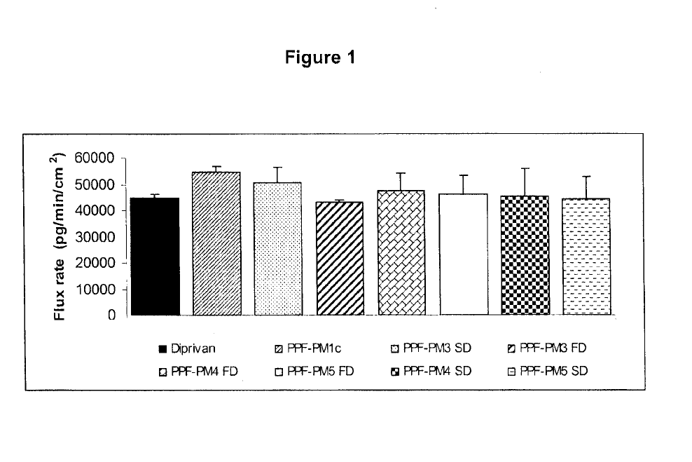

Flux rate of PPF across Caco-2 monolayers is presented in FIG. 1. The results demonstrated that each of the micellar formulations released the PPF for absorption. Levels of translocation were similar for all. The formulations thus demonstrated the ability to translocate propofol across the human endothelial cell monolayers, which in indicative of in vivo bioavailability.

### Example 7

### In Vivo Pharmakokinetic Studies

Rodent Pharmacokinetic Studies

Formulation characteristics of the propofol based products used in the studies reported in this specification are given in Table 7.

TABLE 7
Formulation Characteristics

| | PM1a | PM1b | PM3 FD | PM1c | PM2 |
|---|---|---|---|---|---|
| DLL* | 10% | 20% | 10% | 10% | 10% |
| content | | | | | |
| Cake | White to | White to | White to | White to | White to |
| appearance | yellowish | yellowish | yellowish | yellowish | yellowish |
| Solution | Clear | Clear | opales- | Clear | Clear |
| appearance | yellowish | yellowish | cent | yellowish | yellowish |
| Micelle | 45.5 | 81.8 | 150 | 40.5 | 36.2 |
| size (nm) | | | | | |
| Osmolarity | 364 | 294 | NA | 291 | 250 |
| pH | 7.1 | 7.2 | NA | 7.16 | 7.14 |

DLL: Drug Loading Level
Reconstituted to 1% propofol (10 mg/ml)

In the above Table 7, PM1a stands for a solid product comprising propofol (hereinafter referred to as PPF) loaded to a drug loading level (DLL) of 10%, referred to as P1 which is a PVP-PDLLA having the following characteristics:

% PDLLA: 34.4% (by TGA)

M_{w}=4961

Mₙ=4177

PI=1.2

Similarly, PPF-PM2 stands for a product of the same nature except that propofol is loaded to a drug loading level of 20%.

PM3 FD stands for a solid product comprising PPF loaded to a drug loading level of 10% into a polymer of PEG-PMA having the following characteristics:

PEG-MAA-nBMA: 45-58-26

Mw=13600 (by SLS)

Mn=10709 (by NMR)

PI-1.28

PM1b stands for a solid product having the same polymer composition as PM1a but from a different lot.

PM2 stands for a solid product comprising PPF loaded to a level of 10% into a polymer referred to as P2 which is a PVP-PDLLA having the following characteristics:

% PDLLA: 29.4% (by TGA)

Mw: 4685

Mn=3872

PI=1.2

Pharmacokinetic studies were carried out as follows. Compounds were administered once on Day 1 by a single intravenous (IV, tail vein) bolus injection or per os (PO, oral gavage) to 10-12 week-old female Sprague Dawley rats (body weight ˜170-190 g). Serial blood samples were taken at pre-dose, 1, 5, 10, 20 30 and 60 minutes and 1.5, 2, 4, and 8 hrs post-IV administration and at pre-dose, 5, 10, 15, 20 30 and 60 minutes and 2, 3, 4, 8 and 12 hrs post-PO administration. The blood was immediately transferred in tubes containing heparin as anticoagulant, inverted several times and stored at 4° C. pending further analysis. Propofol concentrations in blood were deternimed using a LC-ESI/MS/MS analytical method (Beaudry et al.; J. Pharm. Biomed. Anal., 39: 411-417, 2005.). Briefly, the analytical procedure for the determination of PPF in rat whole blood consisted in extraction and levels determinations by a HPLC-MS/MS method using Eugenol as internal standard. The assay sensitivity was 20 to 10 000 ng/ml.

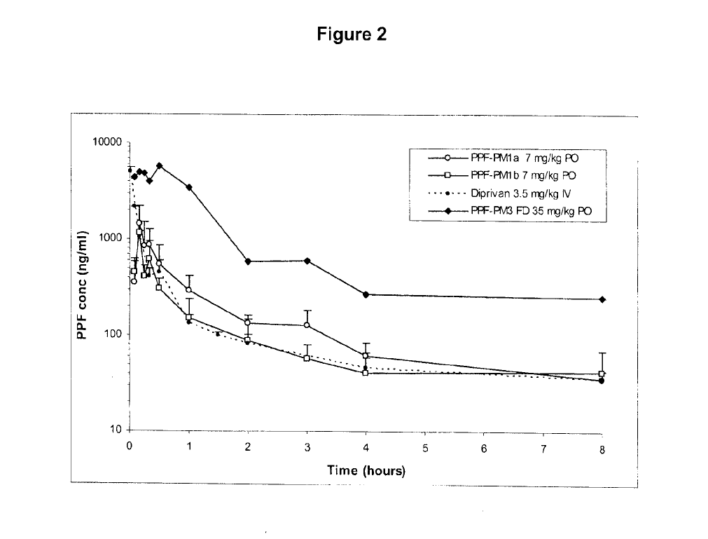

Two pharmacokinetic studies were performed. In the first one, the effect of DLL % was evaluated. Mean concentration-time profiles of PPF in blood following a single IV or PO administration of PM1a, PM2 and PM3 FD are presented in FIG. 2. The pharmacokinetic profiles of PM1a, PM1b and PM3 FD formulations were compared to the commercial formulation: Diprivan® administered IV. Data were normalized to PPF target dose.

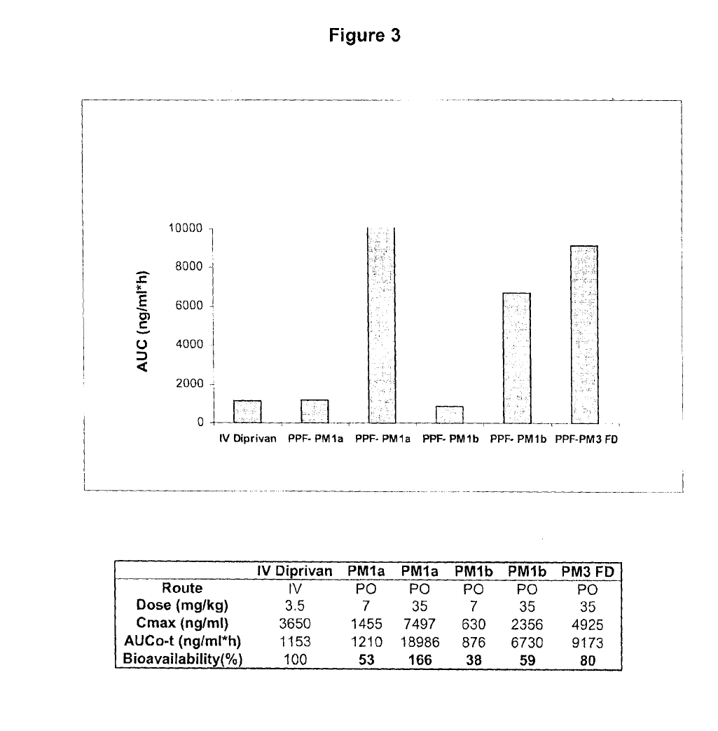

Oral administration of three different oral propofol formulations (viz. PM1a, PM1b and PM3) to Sprague-Dawley rats generated absolute bioavailability values of between 38% and 165% compared to a commercial formulation (Diprivan) given intravenously. F values varied considerably depending on the oral dose administered (FIG. 3). Table 8 gives a summary of the pharmacokinetic parameters obtained from the study.

TABLE 8
Summary of pharmacokinetic parameters in rats

| | IV | | | | | |
|---|---|---|---|---|---|---|
| | Diprivan | PM1a | PM1a | PM1b | PM1b | PM3 |
| Route | IV | PO | PO | PO | PO | PO |
| Dose (mg/kg) | 3.5 | 7 | 35 | 7 | 35 | 35 |
| AUC(ng/ml*h) | 1153 | 1210 | 18986 | 876 | 6730 | 9173 |
| Normalized | 1153 | 605 | 1899 | 438 | 673 | 917.3 |
| AUC (ng/ml*h) | | | | | | |
| Bioavailability | 100 | 52 | 165 | 38 | 58 | 80 |
| (F %) | | | | | | |

As may be seen, there was a pronounced difference between formulations although F values were always considerably higher than those reported for commercially available intravenous formulations given orally. There was also a clear dose/response effect for all formulations. Surprisingly, when formulations were given orally at concentrations of 35 mg/kg absolute bioavailabilities increased versus their corresponding 7 mg/kg dose.

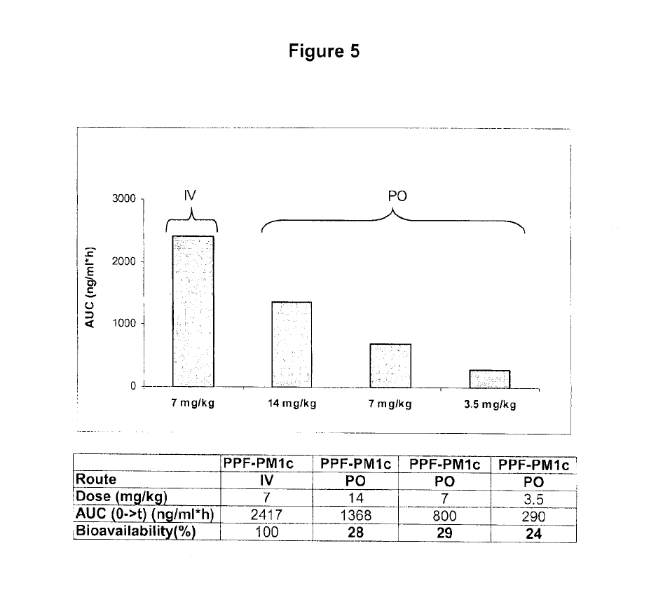

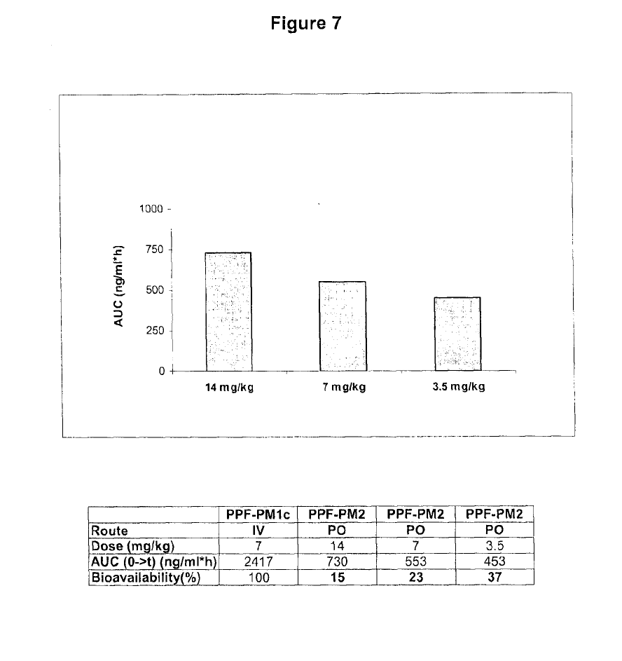

This ‘saturation’ effect was not seen in a second study where lower doses up to 14 mg/kg of a third oral micellar formulation were administered (viz. PM1c—Table 9; FIGS. 5 and 7). While F values were higher than those reported for commercially available intravenous formulations given orally, they were lower than those generated by formulations shown in Table 8 and there was no general increase in bioavailability as dose increased.

Table 9 gives a summary of the pharmacokinetic parameters obtained from the study.

TABLE 9
Summary of pharmacokinetic parameters in rats

| | Diprivan | PM1c | PM1c | PM1c | PM2 | PM2 | PM2 |
|---|---|---|---|---|---|---|---|
| Route | IV | PO | PO | PO | PO | PO | PO |
| Dose (mg/kg) | 7 | 3.5 | 7 | 14 | 3.5 | 7 | 14 |
| AUC (ng/ml*h) | 2417 | 290 | 800 | 1368 | 453 | 553 | 730 |
| Normalized | 2417 | 580 | 800 | 684 | 906 | 553 | 365 |
| AUC (ng/ml*h) | | | | | | | |
| Bioavailability | 100 | 24 | 33 | 28 | 37 | 23 | 15 |
| (%) | | | | | | | |

Minipig Pharmacokinetic Studies

Minipig pharmacokinetic studies were performed in 8-12 kg, 3-6 months-old male Göttingen minipigs as follows:

Test formulations were administered once either orally (oral gavage of micellar formulations) or by intravenous bolus injection (Rapinovet®—a commercially available veterinary formulation of propofol) on day 1 of the study. Serial blood samples were taken;
i) Pre dose and at 1, 5, 10, 20, 30, 60, 120, 240 and 480 minutes post intravenous administration and
ii) Pre dose, 5, 10, 15, 20, 30 and 60, 120, 240, 480 and 720 minutes post-oral administration.

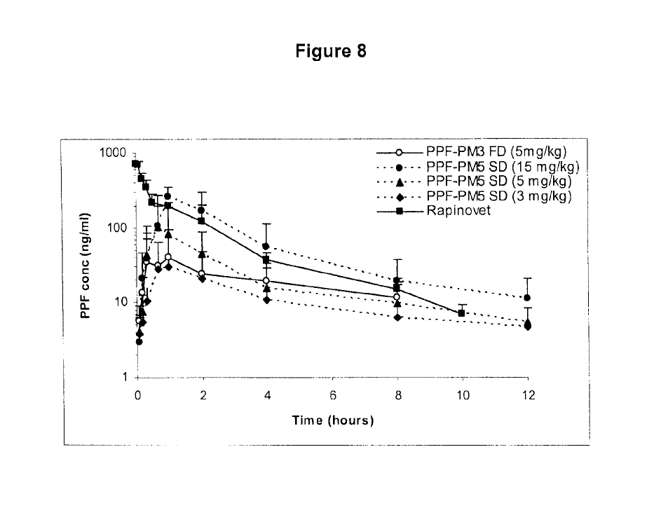

Propofol concentrations were determined using the method described by Beaudry et al. (J. Pharm. Biomed. Anal., 39: 411-417, 2005) Mean propofol concentration-time profiles of PPF following a single IV or PO administration are presented in FIG. 8. Absolute bioavailability values for the oral formulations were generate by comparison with AUC generated by (Rapinovet®) administered intravenously. Absolute bioavailability values of between 14 and 18% were obtained in this model again values considerably higher than those reported for intravenous formulations given by this route: Table 10 summarises the pharmacokinetic data generated.

TABLE 10
Summary of pharmacokinetic parameters in minipigs

| | Rapinovet | PM3 | PM5 | PM5 | PM5 |
|---|---|---|---|---|---|
| Route | IV | PO | PO | PO | PO |
| Dose (mg/kg) | 1 | 5 | 3 | 5 | 15 |
| AUC (ng/ml*h) | 391 | 264 | 193 | 353 | 814 |
| Normalized AUC (ng/ml*h) | 391 | 52.8 | 64 | 71 | 54 |
| Bioavailability(F %) | 100 | 14 | 16 | 18 | 14 |

### Example 8

### Rapid Disintegrating Tablet

A rapid disintegrating tablet (RDT), or ‘wafer’, suitable for sublingual administration of propofol was prepared having the formulation defined below in Table 11.

TABLE 11
Composition of Oral Disintegrating Tablet (Wafer)

| | | Composition (mg/wafer) | |
|---|---|---|---|
| Sr. No. | Ingredient | 07R01801 | 07R01901 |

| | | | | | |
|---|---|---|---|---|---|
| 1 | Propofol | 2.5 | mg | 2.5 | mg |
| 2 | Block copolymer | 22.5 | mg | 22.5 | mg |
| 3 | Contramid ® | 35 | mg | 15 | mg |
| 4 | Mannitol | 35 | mg | 55 | mg |
| 5 | Aspartame | 5 | mg | 5 | mg |
| | Total | 100 | mg | 100 | mg |

Preparation of Propofol Micelles:

Block copolymer was dissolved in 0.1 N NaOH solution and propofol was added to the solution. The mixture was stirred overnight and solution pH was adjusted to 7.5. The Z average diameter of micelles was 158 nm with unimodal size distribution (polydispersity=0.04). Final theoretical propofol concentration of micelles will be 5 mg/ml.

Preparation of RDT:

Aspartame and mannitol were dissolved in above micelle solution and then Contramid® (Labopharm) was dispersed at room temperature.

The above suspension was transferred to the wells of blisters (0.5 ml equivalent to 2.5 mg of propofol) and frozen to −80 C.

The blisters were then lyophilized to form a solid product.

### Example 9

### Propofol Wafers with PEG-PMA Polymer

Propofol Micelles Preparation:

PEG-PMA polymer was dissolved in 0.1 N NaOH solution and propofol was added to the solution. The mixture was stirred overnight and solution pH was adjusted to 7.5. Final theoretical propofol concentration in micelles will be 5 mg/ml.

Preparation of wafers:
1. Aspartame and mannitol were dissolved in above micelle solution and then Contramid was dispersed at room temperature.
2. The above suspension was transferred to the wells of blisters (0.5 ml equivalent to 2.5 mg of propofol) and frozen to −80 C.
3. The blisters were then lyophilized.

The results are summarised in the following Table 12.

TABLE 12

| | | Composition (mg/wafer) | | | |
|---|---|---|---|---|---|
| Sr. No. | Ingredient | Lot 1 | | Lot 2 | |
| 1 | Propofol | 2.5 | mg | 2.5 | mg |
| 2 | PEG-PMA triblock | 22.5 | mg | 22.5 | mg |
| | polymer | | | | |
| 3 | Contramid | 35 | mg | 15 | mg |
| 4 | Mannitol | 35 | mg | 55 | mg |
| 5 | Aspartame | 5 | mg | 5 | mg |
| | Total | 100 | mg | 100 | mg |

| Micelles size (before wafer | 158 nm | | | |
|---|---|---|---|---|
| formulation) | | | | |
| Micelle size from wafers | 159 | nm | 154 | nm |
| (pH 6.8) | | | | |

| | | |
|---|---|---|
| Wafer disintegration time | Less than 10 sec | Less than 10 sec |
| (pH 6.8) | | |

### Example 10

### Propofol Wafers with PVP-PLA Polymer

Propofol Micelles Preparation:

PVP-PLA polymer was dissolved in phosphate buffer pH 6.8 and propofol was added to the solution. The mixture was stirred overnight. Final theoretical propofol concentration in micelles will be 10 mg/ml.

Preparation of Wafers:
1. Aspartame and mannitol were dissolved in above micelle solution and then Contramid was dispersed at room temperature.
2. The above suspension was transferred to the wells of blisters (0.5 ml equivalent to 5 mg of propofol) and frozen to −80 C.
3. The blisters were then lyophilized.

The results are summarised in following Table 13.

TABLE 13

| | | Composition (mg/wafer) | | | |
|---|---|---|---|---|---|
| Sr. No. | Ingredient | Lot 3 | | Lot 4 | |
| 1 | Propofol | 5 | mg | 5 | mg |
| 2 | PVP-PLA polymer | 45 | mg | 45 | mg |
| 3 | Contramid | 35 | mg | 15 | mg |
| 4 | Mannitol | 35 | mg | 55 | mg |
| 5 | Aspartame | 5 | mg | 5 | mg |
| | Total | 125 | mg | 125 | mg |

| Micelles size (before wafer | 26 nm | | | |
|---|---|---|---|---|
| formulation) | | | | |
| Micelle size from wafers | 63 | nm | 38 | nm |
| (pH 6.8) | | | | |

| | | |
|---|---|---|
| Wafer disintegration time | Less than 10 sec | Less than 10 sec |
| (pH 6.8) | | |

All referenced documents are incorporated herein in their entirety.

The above-described embodiments are intended to be examples only. Alterations, modifications and variations can be effected to the particular embodiments by those of skill in the art without departing from the scope of the present disclosure, which is defined solely by the claims appended hereto.

1. A dosage form for non-intravenous administration of a liquid biologically active agent, the dosage form comprising a solid formulation comprising the liquid biologically active agent in intimate association with at least one stabilizing agent.

2. The dosage form according to claim 1, further comprising one or more additives.

3. The dosage form of claim 1 or 2, which, upon hydration, is capable of forming a nanodispersion or micelle loaded with the liquid biologically active agent.

4. The dosage form according to any one of claims 1 to 3, wherein the stabilizing agent comprising at least one amphiphilic copolymer or at least one surfactant.

5. The dosage form according to claim 4, wherein said amphiphilic copolymer comprises a linear, branched or star-shaped block polymer.

6. The dosage form according to claim 4 or 5 wherein the amphiphilic polymer includes a hydrophilic segment is selected from poly(ethylene oxide), poly(N-vinylpyrrolidone), poly(N-2-hydroxypropylmethacrylamide), poly(2-ethyl-2-oxazoline), poly(glycidol), poly(2-hydroxyethylmethacrylate), poly(vinylalcohol), polymethacrylic acid derivatives, poly(vinylpyridinium), poly((ammoniumalkyl)methacrylate), poly((aminoalkyl)methacrylate) and combinations and derivatives thereof; and a hydrophobic segment selected from the group comprising a poly(ester), poly(ortho ester), poly(amide), poly(esteramide) poly(anhydride), poly(propylene oxide), poly(tetrahydrofuran), polystyrene, polymethacrylate, polyacrylate, polymethacrylic acid, polyacrylic acid and combinations and derivatives thereof.

7. The dosage form according to claim 6, wherein said hydrophobic segment comprises a poly(ester) selected from the group consisting of poly(ε-caprolactone), poly(lactide), poly(glycolide), poly(lactide-co-glycolide), poly(hydroxyl-alkanoates), poly(β-malic acid), and combinations and derivatives thereof.

8. The dosage form according to any one of claims 4 to 8, wherein said amphiphilic copolymer is a PVP-PDLLA or PEG-PMA copolymer.

9. The dosage form according to claim 8, wherein said amphiphilic copolymer is a diblock or triblock PEG-PMA copolymer.

10. The dosage form according to claim 9, wherein the PEG-PMA copolymer is an EG-MAA-BMA copolymer.

11. The dosage form according to claim 10, wherein the EG-MAA-BMA copolymer has the following composition: EG₍₂₀₋₅₀₀₎-MAA₍₅₋₅₀₀₎-BMA₍₅₋₅₀₀₎.

12. The dosage form according to claim 11, wherein the EG-MAA-BMA hays one of the following compositions: EG₍₄₅₎-MAA₍₆₃)-BMA₍₂₈₎; EG₍₄₅₎-MAA₍₆₄₎-BMA₍₃₄₎; or EG₍₄₅₎-MAA₍₅₄₎. BMA₍₂₆₎.

13. The dosage form according to claim 8, wherein said amphiphilic copolymer is a PVP-PDLLA copolymer.

14. The dosage form according to claim 1 wherein said stabilizing agent comprises a surfactant.

15. The dosage form according to claim 14, wherein said surfactant is selected from the group comprising lauryl sulphate, hexadecyl pyridinium chloride, polysorbates, sorbitans, poly(oxyethylene) alkyl ethers, poly(oxyethylene) alkyl esters and combinations thereof.

16. The dosage form according to any one of claims 1 to 15, which is prepared from a solid formulation comprising the liquid biologically active agent in intimate association with at least one stabilizing agent, and one or more additives.

17. The dosage form according to any one of claims 1 to 16, wherein the solid formulation is obtained by drying a mixture of the stabilizing agent, the liquid biologically active agent, and at least one solvent therefore, in such a manner as to form the intimate mixture of the liquid biologically active agent and the stabilizing agent.

18. The dosage form according to claim 17, wherein the drying is lyophilization or freeze-drying.

19. The dosage form according to claim 17, wherein the drying results in a powder.

20. The dosage form according to claim 19, wherein the drying is spray-drying or fluid bed-drying.

21. The dosage form according to any one of claims 1 to 20, wherein the liquid biologically active agent is present in the solid formulation in a therapeutically effective amount.

22. The dosage form according to any one of claims 1 to 21, wherein the liquid biologically active agent is present in the solid formulation in an amount between about 1 wt % and about 80 wt %, between about 1 wt % and about 60 wt %, between about 5 wt % and about 40 wt %, between about 5 wt % and about 30 wt %, between about 10 wt % and about 30 wt %, between about 10 wt % and about 20 wt %, between about 0.1 wt % and 5 wt %, between about 1 wt % and about 5 wt %.

23. The dosage form according to any one of claims 1 to 21, wherein the solid formulation is present in the dosage form in an amount from about 1 wt % to about 99 wt %, from about 5 wt % to about 85 wt %, from about 5 wt % to about 60 wt %, 5 wt % to about 40 wt %, between about 5 wt % to about 30 wt %, between about 10 wt % to about 30 wt %, between about 10 wt % to about 20 wt %, between about 0.1% to 5%, between about 1 wt % to about 5 wt %, between about 20 wt % to about 60 wt %.

24. The dosage form according to any one of claims 1 to 21, wherein the biologically active agent is present in the dosage form in an amount from about 0.01 wt % to about 80 wt %, 0.01 wt % to about 50 wt %, from about 1 wt % to about 20%, from about 1 wt % to about 15 wt %, from between about 2 wt % to about 10 wt %, between about 1 wt % to about 5 wt %, between about 5 wt % to about 10 wt %, or between about 10 wt % to about 20 wt %.

25. The dosage form according to any one of claims 1 to 24, wherein, upon administration to a subject, the dosage form provides a bioavailability sufficient for achieving therapeutic efficacy.

26. The dosage form according to claim 25, wherein the bioavailability of the active agent is at least about 2%, 5%, 10%, 15%, 20%, 25%, 30%, 35%, 40%, 45%, 50%, 55%, 60%, 65%, 70%, 75%, 80%, 85%, 90%, 95%, 100%, or higher.

27. The dosage form according to claim 25, wherein the dosage form exhibits an increase in bioavailability of at least 10% compared to same-route administration of the biologically active agent in the absence of the stabilizing agent.

28. The dosage form according to claim 25, wherein the dosage form exhibits a relative bioavailability of at least 100%, 110%, 120%, 150%, 200%, 500%, 700%, or 1000%.

29. The dosage form according to claim 25, wherein the dosage form exhibits a absolute bioavailability of at least 10%.

30. The dosage form according to claim 25, wherein the bioavailability of the active agent is increased by at least about 1.5-fold, 2-fold, 3-fold, 5-fold, 10-fold, 15-fold, 20-fold, 30-fold, 40-fold, 50-fold, 75-fold, 100-fold, or higher, in the presence of the stabilizing agent.

31. The dosage form according to claim 25, wherein the bioavailability of the active agent is increased by at least about 1.5-fold to about 40-fold, from about 2-fold to about 35-fold, from about 5-fold to about 30-fold, in the presence of the stabilizing agent.

32. The dosage form according to any one of claims 1 to 31, wherein the solid formulation has a drug loading level (DLL) of up to about 5%, 10%, 15%, 20%, 25%, 50%, 60%, 70%, 80%, or higher.

33. The dosage form according to any one of claims 1 to 31, wherein the solid formulation has a drug loading level (DLL) from about 1% to about 80%, from about 10% to about 80%, or from about 20% to about 60%.

34. The dosage form according to any one of claims 3 to 33, wherein, the micelles have a diameter less than about 500 nm, such as, between about between about 5 nm to 500 nm, 10 nm to 500 nm, 10 nm to 400 nm, 20 nm to 300 nm, or 20 nm to 200 nm.

35. The dosage form according to any one of claims 3 to 34, wherein the stabilizing agent has a CAC below about 100 mg/L, below about 50 mg/L, below about 25 mg/L, below about 10 mg/L, or below about 5 mg/L.

36. The dosage form according to any one of claims 3 to 34, wherein the stabilizing agent has a CAC in the range of about 0.1 mg/L to about 1000 mg/L, about 0.1 mg/L to about 100 mg/L, about 0.1 mg/L to about 50 mg/L, about 0.1 to about 25 mg/L, about 0.1 to about 10 mg/L, or about 0.1 to about 5 mg/L.

37. The dosage form according to any one of claims 1 to 36, wherein the liquid biologically active agent is hydrophobic or amphiphilic.

38. The dosage form according to claim 37, wherein the liquid biologically active agent is selected from the group consisting of propofol, quinaldine, methoxyflurane, nicotine, phytonadione, methoxyflurane, dinoprost tromethamine, and mesoprostol, or a prodrug or derivative thereof.

39. The dosage form according to any one of claims 1 to 38, which is suitable for oral, sublingual, intranasal, intrapulmonary, rectal, urethral, vaginal, ocular, otic or topical administration.

40. The dosage form according to claim 39, which is suitable for oral administration.

41. The dosage form according to claim 40 wherein the dosage form exhibits an absolute bioavailability of at least 10%.

42. The dosage form according to claim 39, which is suitable for sublingual administration.

43. The dosage form according to any one of claims 1 to 42, wherein the dosage form is in the form of a tablet, caplet, capsule, sachet, solution, suspension, emulsion, cream, gel, film, lozenge, chewing gum, paste, ointment, drop, spray, aerosol inhaler, dry powder inhaler, suppository, pessary, or enema.

44. The dosage form according to any one of claims 2 to 43, wherein the additive is one or more of a carrier, a bulk forming agent, a cryoprotectant, a lyoprotectant, a binder, a flavoring agent, a taste masking agent, a coloring agent, an odorant, a buffer, a preservative, a diluent, a dispersant, a surfactant, a disintegrant, or an additional stabilizer.

45. The dosage form according to claim 43, wherein the tablet is a rapid disintegrating tablet (RDT).

46. The dosage form according to claim 44, wherein the RDT comprises a disintegrant or disintegrating matrix to facilitate rapid release of the solid formulation from the dosage form.

47. The dosage form according to claim 45, wherein the disintegrating matrix is a starch or a hydrogel.

48. The dosage form according to claim 46, wherein the starch is a cross-linked high amylose starch, such as, Contramid.

49. The dosage form according to any one of claims 45 to 48, wherein the RDT additionally comprises a sugar, such as, mannitol, trehalose, maltodextran.

50. The dosage form according to any one of claims 1 to 49, which is an instant release dosage form, an immediate release dosage form, or a controlled release dosage form.

51. The dosage form according to claim 50, wherein the controlled release is sustained release, and wherein the dosage form releases the liquid biologically active agent over a period of about 45 minutes to about 24 hours.

52. The dosage form according claim 51, wherein the controlled release is sustained release, and wherein the dosage form releases the liquid biologically active agent over a period of at least about 4 hours, at least about 8 hours, at least about 12 hours, at least about 16 hours, at least about 20 hours, or at least about 24 hours.

53. The dosage form according to claim any one of claims 1 to 52, wherein the liquid biologically active agent is propofol or a derivative or prodrug thereof.

54. The dosage form according to claim 53, wherein the liquid biologically active agent is propofol.

55. The dosage form according to any one of claims 1 to 54, wherein the solid formulation comprises between about 10 wt % and about 30 wt % propofol.

56. The dosage form according to claim 54 or 55, wherein, upon oral administration, the absolute bioavailability of propofol is at least about 10%, between about 15% and about 165%, between about 15% and about 100%, between about 15% and about 80%, or between about 20% and about 80%.

57. The dosage form according to any one of claims 53 to 56 for use in the treatment or prevention of a disease or condition of the central nervous system.

58. The dosage form according to claim 57 wherein the disease or condition of the central nervous system is headache, emesis, nausea, or pain.

59. The dosage form according to any one of claims 53 to 56 for inducing anaesthesia or sedation in a subject in need thereof.

60. The dosage form according to any one of claims 1 to 56 for use in the manufacture of a medicament.

61. Use of the dosage form according to any one of claims 53 to 56 in the treatment or prevention of a disease or condition of the central nervous system.

62. Use of the dosage form according to any one of claims 53 to 56 in the manufacture of a medicament for the treatment or prevention of a disease or condition of the central nervous system.

63. Use of a solid formulation as defined in any one of claims 1 to 56 in the manufacture of a non-intravenous dosage form for the treatment or prevention of a disease or condition of the central nervous system.

64. Use of a solid formulation comprising an intimate mixture of propofol and at least one amphiphilic copolymer in the manufacture of a non-intravenous dosage form for the treatment or prevention of a disease or condition of the central nervous system.

65. The use according to any one of claims 61 to 63, wherein the condition of the central nervous system is headache, nausea, emesis, or pain.

66. A solid formulation comprising an intimate mixture of propofol and at least one stabilizing agent, for use in the manufacture of a non-intravenous dosage form for the treatment or prevention of headache, nausea, emesis, or pain.

67. A method or treating a disease or condition, comprising administering to a subject in need thereof a therapeutically effective amount of a non-intravenous dosage form as defined in any one of claims 1 to 56.

68. The method according to claim 67, wherein the route of administration is oral, sublingual, intranasal, intrapulmonary, rectal, urethral, vaginal, ocular or topical administration.

69. The method according to claim 68, wherein the route of administration is oral administration.

70. The method according to claim 68, wherein the route of administration is sublingual administration.

71. The method according to any one of claims 67 to 70, wherein the disorder or condition to be treated is disease or condition of the central nervous system.

72. The method according to claim 71, wherein the condition is headache, nausea, emesis or pain, and wherein the dosage form is as defined in any one of claims 53 to 56.

73. The method according to claim 72, wherein the headache is intractable migraine headache.

74. The method according to claim 72, wherein the pain is neuropathic pain.

75. The method according to claim 74, wherein neuropathic pain is post-herpetic neuralgia, peripheral neuropathy, trigeminal neuralgia, lower back pain, painful diabetic neuropathy, HIV-related neuropathic pain, cancer-related pain, or fibromyalgia.

76. A method of treating or preventing headache, nausea, emesis or pain, comprising administering to a subject in need thereof a therapeutically effective amount of a non-intravenous dosage form comprising a solid formulation, and, optionally, one or more additives, the solid formulation comprising an intimate mixture of propofol and at least one amphiphilic copolymer, wherein, upon hydration, micelles loaded with the propofol are formed.

77. A commercial package or kit comprising a non-intravenous dosage form as described in any one of claims 1 to 56, together with one or more instructions for use in the treatment or prevention of a disease or condition.

78. A commercial package or kit comprising a non-intravenous dosage form as described in any one of claims 53 to 56, together with one or more instructions for use in the treatment or prevention of headache, nausea, emesis, or pain.

79. A method for the preparation of a dosage form for non-intravenous administration of a liquid biologically active agent which comprises:
providing a first mixture of at least one stabilizing agent in at least one solvent, under conditions to achieve micelle or nanodispersion formation,
providing a second mixture by mixing said first mixture and at least one liquid biologically active agent to load said micelle or nanodispersion with said liquid biologically active agent,
removing the solvent from said second mixture to form a solid formulation; and
optionally, adding one or more additives suitable to prepare the non-intravenous dosage form.

80. The method of claim 79, wherein the solvent is removed by drying.

81. The method of claim 80, wherein the drying involves spray drying or drying in a fluid bed.

82. The invention as hereinbefore described.
